(19) United States
(12) Patent Application Publication
(10) Pub. No.: US 2015/0323525 A1
(43) Pub. Date: Nov. 12, 2015

(54) METHODS AND MEANS FOR DETECTING CELLS USING SURFACE PLASMON RESONANCE

(71) Applicants: STICHTING SANQUIN BLOEDVOORZIENING, Amsterdam (NL); IBIS TECHNOLOGIES B.V., Enschede (NL)

(72) Inventors: Gestur Vidarsson, Maarssen (NL); Richard Bernardus Maria Schasfoort, Enschede (NL)

(21) Appl. No.: 14/654,214

(22) PCT Filed: Dec. 19, 2013
(86) PCT No.: PCT/NL2013/050927

§ 371 Date: Jun. 19, 2015

(30) Foreign Application Priority Data
Dec. 19, 2012 (EP) 12198052.8

(51) Int. Cl. G01N 33/543

(52) U.S. Cl. CPC G01N 33/54373; G01N 2333/705

(57) ABSTRACT

The invention relates to means and methods for detecting a cell surface molecule in a cell sample. The invention further relates to a method for blood group typing and screening and to SPR sensors and SPR measuring systems suitable for use in such methods. The method includes steps of allowing a liquid cell sample to flow to and along the sensor surface, temporarily reducing the shear rate of the liquid sample to allow cells in the sample to sediment by binding to a binding compound immobilised on a metal film on the sensor surface, and removing unbound cells or non-specifically bound cells or fragments thereof. Moreover, the ratio of the magnitude of the specific total signal response to the signal response during sedimentation is determined.

The invention relates to analysis of cell surface molecule expression. More specifically, the invention relates to methods for detection of cell surface molecules using surface plasmon resonance.

Studies focusing on retrieving qualitative and quantitative information on cell membrane antigens are relevant for various applications. Most widely used technologies for cell surface antigen determination are flow cytometry and fluorescent microscopy. For both techniques, antibodies targeting the cell membrane antigens are fluorescently labelled and the fluorescent signals obtained from the cells correspond to the density of the antigens. To assess multiple antigens simultaneously, antibodies are labelled with different fluorochromes. In this manner multiplexing of four to six antigens can be performed routinely. Multiplex typing is also possible using genotyping approaches, however those methods are hampered by the presence of null-alleles, resulting in false positive results and, similar to cytometrical methods, are accompanied with relatively high costs. The use of a label free method such as surface plasmon resonance (SPR) imaging may overcome these issues. SPR imaging technologies are well known. With such technology shifts in SPR angle due to biomolecular interactions at a sensor surface are measured label free and in real time

Although label-free analysis of biomolecular interactions by SPR is widely used for determinations of binding kinetics, the label free binding of cells to ligand-spotted surfaces have not yet been clinically or commercially used because SPR methods that allow practical and accurate typing of whole cells for clinical or research purposes have thus far not been developed. The problems encountered for cellular detection in SPR are, for instance, that suspended cells under shear in SPR instruments bounce from the wall of flow channels due to the laminar flow profile and interaction with the sensor surface will be hampered because of the size of the cell in relation to the thickness of the stagnant layer under such shear conditions. Further, for practical reasons, most commercial SPR instruments are configured with optics on top of the fluidics to avoid leakage of liquid into the optical compartment of the instrument, hampering contact between whole cells and the sensor surface. In addition, cell interactions to immobilized antibodies or antigens do not show 1:1 binding kinetics because of the multiple antigens present on the cell surface. Avidity based models using serial dilutions of the ligand densities should therefore be applied for the number of interactions and for qualifying the affinity of the cell antigen antibody interaction. Another possible difficulty in using SPR for whole cell measurements is that a cell is many times (>20×) larger than the penetration depth of the evanescent field so that only a small part of the cell is within the decaying evanescent sensing area. In addition, responses due to cell adhesion will depend on the orientation and packing density of bound cells. A non-linear response is to be expected close to saturation levels of the surface area. Finally, cells and cellular particles are expected to clog valves and tiny flow channels in microfluidic cartridges.

WO 2004/088318 describes analysing cell samples using SPR inhibition assays. In such inhibition assays a fixed concentration of a specific antibody (such as a mouse antibody) with affinity to a cell surface molecule is mixed with a sample containing cells. The mixture is subsequently passed over a sensor surface to which an antibody against the specific antibody (such as a rabbit anti-mouse antibody) is immobilized. Antibodies in the sample, which did not bind to the cell surface molecules, are detected as they bind to the antibodies immobilized on the sensor surface. Thus, direct binding of cells to the sensor surface does not occur.

Quinn et al. (J Immunol Methods. 1997; 206(1-2):87-96) describes detection of a blood group antigen on red blood cells using a BIAcore™ SPR system (Biacore AB, Uppsala Sweden). Concanavalin A and a blood group specific IgM antibody are immobilized onto the sensor surface in a dextran matrix in order to bind red blood cells. Quinn et al. report difficulties with regeneration of the sensor surface which resulted in an accumulative loss in surface binding activity after each binding-regeneration cycle and a dramatic decrease in activity after regeneration of a red blood cell-saturated surface. Another disadvantage of the method of Quinn et al. is that an assay takes a considerable time to finish due to the low flow speed of 5 μl/min which is necessary to allow binding. Red blood cell samples thus needed to be passed over the sensor surface for at least 15 minutes. Further, elution of bound red blood cells also took at least 15 minutes. As a result, a single SPR measurement of Quinn et al. takes up to one hour to be completed. Finally, determining binding of low concentrations of red blood cells was not possible due to low fractional coverage of the sensor surface.

It is an object of the present invention to provide methods and means that enable fast, sensitive and accurate label-free cell detection and cell typing that overcome the disadvantages indicated above. It is further an object of the present invention to provide methods and means that provide, for example, an alternative to conventional blood group typing technologies.

The present inventors found that a fast, sensitive and accurate SPR method for typing cell surface molecules is achieved when during flow of a sample containing cells along an SPR sensor surface contact between cells and the sensor surface is temporarily enhanced and flow is not constant as in conventional SPR methods. In particular is was found that, after flow of sample along the sensor surface was initiated, stopping the flow allowing diffusion and/or sedimentation of cells resulted in an improved method of analysing cell samples for expression of surface molecules as described in the Examples.

It was found that when conventional SPR methods are used for analysing samples comprising whole cells, when cells are flowing in sample tubing and in flow chambers the laminar flow therein will deplete the stagnant layer of cells close to the wall. So the laminar velocity profile is localized in a similar dimension as the size of the cells, and the cell will therefore always bounce from the stagnant layer and a part of the cell, and consequently the whole cell, will be dragged into the higher velocity of the liquid. In other words, after a lateral transport of the cell suspension over the SPR sensor surface, the cells are not in direct contact with these surfaces, but will stay in the middle of the stream. The shear rate determines the effect of dragging into the middle stream and pulling of the wall and this is the parameter which takes the geometries and flow velocities into account. Therefore the cells will essentially not be in contact with any binding compounds immobilized on the sensor surface and binding of cells to these compounds will not be observed above a certain value of the shear rate. The invention provides an economical high-throughput so called multiplex method for whole cell typing and antibody screening which reduces the hands-on time and costs and provides automation in cell typing and screening procedures, and increases blood and organ donor-recipient matching if expression of blood group antigens is analyzed.

Accordingly, the invention provides a method for detecting a cell surface molecule comprising:
a) providing a sensor comprising a sensor surface, said sensor surface comprising a transparent element covered with a metal film of at most 100 nm thick and at least one binding compound immobilized to said metal film,
b) allowing a liquid cell sample to flow to and along the sensor surface,
c) temporarily reducing shear rate of said liquid sample, preferaby to allow cells in said liquid sample to bind said binding compound,
d) removing unbound cells or non-specifically bound cells, and
e) determining a presence or absence of a change in refractive index of light incidence at the sensor surface.

A “cell surface molecule” as used herein refers to a molecule that is expressed on the surface of a cell so that at least part of the molecule is extracellularly. Examples of cell surface molecules are receptors including GPCR's, integrins, proteins and antigens. The presence or absence of specific cell types can be determined based on the presence of one or more specific cell surface molecules such as receptors and/or antigens.

A “sensor surface” as used herein refers to the surface of a metal film, preferably gold or silver, covering a transparent element such as a prism and comprising a plurality of spots to which binding compounds can be immobilized. The sensor surface optionally includes chemical linking layers including gel type layers. An example of a typical sensor surface is a gold surface of about 50 mm² comprising 48-96 spots.

As used herein “temporarily” refers to a limited period of time of the total time of an SPR measurement. The duration of such period depends on cell type present in the sample, on the shear rate and on the manner by which contact between cells and the cell surface is enhanced.

As used herein the term “specific for” or “specifically binding” refers to the interaction between an antibody and its epitope, indicating that said antibody preferentially binds to said epitope, or between a protein and its target, indicating that said protein preferentially binds to said target. Thus, although the antibody or protein may non-specifically bind to other antigens or amino acid sequences, the binding affinity of said antibody for its epitope and of said protein to for its target is significantly higher than the non-specific binding affinity of said antibody or protein for any other antigen or amino acid sequence.

“Enhancing” as used herein refers to increasing the level of contact between cells and the binding compound(s). Thus, with respect to enhancing contact between cells and immobilized binding compound, the term “enhancing” indicates that the number of cells that are in contact with multiple immobilized binding compound, or the cellular penetration into the evanescent field of the sensor surface, is increased, preferably more than one fold, preferably at least two-fold, three-fold, four-fold, ten-fold or 50-fold.

A “plurality” as used herein refers to at least two. With respect to binding compounds, such as antibodies or antigens, a plurality as used herein preferably refers to at least three, more preferably at least four, more preferably at least five. The amount of different binding compounds that can be immobilized on a sensor surface is limited only by the size of the sensor surface. For instance, a sensor surface according to the present invention may comprise up to 96 or more spots, each spot comprising a different binding compound.

“Shear rate” as used herein refers to the relative velocities of layers of a liquid under laminar flow. The effect of flow of a liquid sample and cell adhesion and detachment of cells in the liquid sample can be described using the shear rate. The SI unit of measurement for shear rate is sec⁻¹, expressed as “reciprocal seconds” or “inverse seconds”. The shear rate at the inner wall of a Newtonian fluid flowing within a pipe is:

$$\dot{γ} = \frac{8 v}{d}$$

where:

\dot{γ}=The shear rate, measured in reciprocal seconds.

v=The linear fluid velocity.

d=The inside diameter (ID) of the pipe.

The linear fluid velocity v is related to the volumetric flow rate Q by:

$$v = \frac{Q}{A}$$

where A is the cross-sectional area of the pipe, which for an inside pipe radius of r is given by:

A=πr²

thus producing:

$$v = \frac{Q}{π {r}^{2}}$$

Substituting the above into the earlier equation for the shear rate of a Newtonian fluid flowing within a pipe, and noting (in the denominator) that d=2r:

$$\dot{γ} = \frac{8 v}{d} = \frac{8 ( \frac{Q}{π {r}^{2}} )}{2 r}$$

which simplifies to the following equivalent form for wall shear rate in terms of volumetric flow rate Q and inner pipe radius r:

$$\dot{γ} = \frac{4 Q}{π {r}^{3}}$$

Equation shear rate with respect to flow rate in pipes.
In the SPR system the tubing as inner diameter of 1 mm and the above equation can be applied to determine the shear rate. However the flow chamber with the inlet and outlet has a different geometry and in the paper D P Bakker, et al. (Appl Environ Microbiol. 2003; 69(10): 6280-6287) the shear rate for the applied flow chamber as part of this invention can be calculated. Because typically the applied flow chamber has a similar cross sectional area (A) as the connected tubing, the shear rate in the flow chamber can be considered similar (but not equal) to the shear rate in an 1 mm tubing (pipe). According to the above equation shear rate with respect to flow rate in pipes typically flow rates of 10 μl/s for the applied ID tubing of 1 mm give values of shear rate ˜100 s⁻¹.

An SPR system typically comprises a source of polarized light, a sensor comprising a transparent element such as a prism covered with a thin metal film and linking layer, a flow chamber, a system for controlling transport of liquid, and an optical unit for detecting reflected light. The SPR principle is based on the excitation of surface plasmons in a thin layer of a metal such as gold or silver, using polarized light. Polarized light from the prism is reflected from the metal surface, and at a certain angle of light incidence the excitation of resonance in the metal film results in intensity and phase changes in the reflected light beam. An evanescent field is generated which travels in a direction perpendicular to the surface, i.e. in the direction of the liquid sample. As a result of excitation of surface plasmons the incident light is adsorbed, resulting in a decrease in the intensity of the reflected light. For each wavelength and the corresponding effective refractive index ratios between both sides of the metal layer, there is a specific angle at which a minimum in reflectivity is observed, the SPR angle. This angle increases with decreasing wavelength. When interactions occur on the metal surface within the range of the penetration depth of the evanescent field while at the glass phase the refractive index remains constant a change in refractive index of the dielectric liquid sample occurs. This change in refractive index causes a change in the angle at which SPR occurs. This change in SPR angle is recorded as a shift in the SPR angle with time, resulting in SPR sensorgrams. The change in SPR angle of reflected light is directly proportional to the binding of analyte on the metal surface of the prism.

One of the most common geometries of SPR systems is the Kretschmann configuration. In this configuration the incident light comes from the high refractive index material (e.g. a prism) and reflects at the metal surface without travelling through the liquid. Surface plasmon resonance is not only suited to measure the difference between these two states, but can also monitor the change in time if the shift of the resonance angle is followed in time Apart from kinetic analysis, SPR measurements can also be used for quantitative analysis, i.e. for determination of the concentration of the analyte in a sample.

The detection format for SPR measurements is based on a number of characteristics, such as the size of the analyte molecules, the binding characteristic between analyte and immobilized binding compounds, concentration of the analyte to be measured, and sample matrix including temperature. For direct detection, a binding compound (e.g., antibody) is immobilised on the SPR sensor surface. Analyte in the sample binds to the antibody, producing a refractive index change, which is detected by the SPR sensor. In principle, the direct detection allows binding of low molecular weight compounds down to 200 Dalton, depending on noise levels of the instrument and ligand surface capacity of the sensor surface. The sandwich detection format can be used for smaller analytes with more than two specific epitopes for enhancing the specificity and reduction of non-specific binding of interacting compounds. Here, the sensor surface with bound analyte is additionally incubated with a second detection antibody. In competition assays, the sensing surface is coated with a binding compound binding to the analyte. When analyte molecules conjugated to a large molecule are added to the sample, the analyte and its conjugated analogue compete for a limited number of binding sites on the sensor surface. The binding response is inversely proportional to the analyte concentration. In inhibition assays a fixed concentration of a specific antibody (such as a mouse antibody) with affinity to an analyte is mixed with a sample. The mixture is subsequently passed over a sensor surface to which an antibody against the specific antibody is immobilized. Antibodies in the sample, which did not bind to the analyte, are detected as they bind to the antibodies immobilized on the sensor surface. The binding response is thus inversely proportional to the concentration of the analyte.

A method of the invention preferably relates to detecting the presence or absence of a cell surface molecule.

In step a) of a method of the invention a sensor comprising a sensor surface is provided. The sensor surface comprises a transparent element covered with a metal film of at most 100 nm thick for generating surface plasmon resonance. The incident light is directed at a transparent element which is at least partially covered by a metal film. The transparent element has a refractive index greater than that of the dielectric layer formed by the sample at the metal surface. Preferred transparent elements are a prism, and optical fibres. Most preferably, the transparent element is a prism, which can be made of glass, quartz or synthetic material, preferably glass. Thus in a preferred embodiment, said transparent element is a glass prism. Specific examples of suitable prisms are those of K5, BK7, SF10, SF11 glass, for instance triangular and hemispherical prisms.

The metal film is capable of exhibiting surface plasmon resonance. Preferably said metal is gold or silver, most preferably gold. The metal film should be sufficiently thin to allow the evanescent wave to penetrate through the metal film and to couple with the surface plasmons at the outer surface of the metal film. The metal film therefore has a thickness of at most 100 nm Preferably said metal film, preferably gold or silver, most preferably gold, has a thickness of between 20 and 80 nm, more preferably between 40 and 60 nm, most preferably between 45 and 55 nm. In specific embodiment, the thickness of the metal film is about 45, 46, 47, 48, 49, 50, 51, 52, 53, 54 or 55 nm.

The liquid passing over the sensor surface flows through a so-called flow chamber. The flow chamber is in liquid contact with the sensor surface to allow liquid passing through the flow chamber to be in contact with the sensor surface. The size of an exemplary flow chamber is 6 (w)×10 (1)×0.2 (h) mm. A system for allowing transport of liquid, such as sample or (regeneration) buffer, through the flow chamber and over the sensor surface is coupled to the flow chamber. Such system for instance comprises a tubing system, one or more pumps, means for injecting liquid, such as sample or buffer, and one or more valves for regulating flow rate and direction. An SPR sensor and a flow chamber may form an exchangeable cartridge.

Following analysis of a cell sample, the sensor surface is preferably regenerated to detach and remove cells or antigens bound to the binding compounds immobilized on the sensor surface. Immobilization of binding compounds to the sensor surface is preferably not affected by regeneration or effected to only a minor extent. Regeneration is for instance performed by allowing a regeneration buffer to flow along the sensor surface. Suitable examples of regeneration buffers include acidic or basic solutions, for example 1-100 mM glycine-HCl pH 2 (pH1-pH3), phosphoric acid pH 1-pH 3 and 1-100 mM NaOH (pII 11-pII 13). For cellular regeneration, a combination of these buffers together with detergents, for example Tween, saponine, or deoxycholate, are highly suitable for dissolving the cellular membranes for even a more optimal regeneration process. Following regeneration, a subsequent measurement in accordance with the invention can be performed.

In step b) of a method of the invention a liquid cell sample is allowed to flow to and along the sensor surface. This step serves essentially to transport the sample to the flow chamber to allow contact between the liquid sample and the sensor surface. Contrary to conventional SPR methods, binding of cell surface molecules in the sample to binding compound immobilized on the metal film of the sensor surface does not substantially occur during this step. Therefore, flow rate and liquid velocity are not critical in step b). The higher the flow rate during step b), the faster the cell reaches the flow chamber, and the higher the overall speed of a measurement performed in accordance with the invention. Therefore, in a preferred embodiment, the flow rate in step b) is at least 10 μl/min, preferably at least 50 μl/min, more preferably at least 5 μl/s, more preferably at least 20 μl/s, more preferably at least 100 μl/s, such as between 5 μl/s and 150 μl/s. The shear rate in step b) is preferably at least 50 s⁻¹, more preferably at least 500 s⁻¹

Step d) of a method of the invention comprises removing unbound cells or non-specifically bound cells from the sensor surface. Sedimented cells which are not recognized and bound by the immobilized binding compound are preferably washed away in step d). The shear rate that allows removal of unbound cells or non-specifically bound cells depends on the cell type present in the liquid sample, and on the size of the specific cells. For instance, unbound or non-specifically bound large cells, such as monocytes and several tumour cells, will be more readily removed, i.e. at a lower flow/shear rate, than smaller cells such as red blood cells and platelets and cell fragments such as microvesicles. In principle, adhering cells capable of cell-spreading or fragments capable of spreading (e.g. white blood cells, endothelial cells, fibroblasts, platelets or microvesicles) bind non-specifically to the surface but cannot easy be removed by applying high shear rates regenerating the specific interaction. However specific treatment of such cells, e.g. with fixing compounds like paraformaldehyde, prevent the adherence and the treated cells can be handled similar as non-adhering cells like red blood cells. Further, the flow/shear rate necessary may depend on the type of binding compound and the amount of cell surface molecules per cell that bind to immobilized binding compounds. In addition to the cell type and cell size and binding compound, the inner diameter of the flow channel wherein the liquid sample is located influences the flow rate that allows removal of unbound cells or non-specifically bound cells, but does not influence specific binding of cells to the sensor surface or the integrity of such specifically bound cells. Hence, the shear force depends on the liquid velocity profile, which is determined by both the flow rate and the form and size of the flow channel. For instance, if the size of the flow channel is decreased while maintaining a constant flow rate, the liquid velocity and the shear rate is increased The flow rate at a given size of the flow channel during step d) can be tuned to get a critical shear rate for disrupting cells from the sensor surface in accordance with the invention. The shear rate at step d) of a method of the invention should not be too high, because this may result in disruption of the cells bound to the sensor surface. For instance, for a flow channel having an inner diameter of about 1 mm, the flow rate of the liquid sample in step d) is preferably between 10 μl/min and 100 μl/second, more preferably between 1 μl/second and 70 μl/second, even more preferably between 5 μl/second and 50 μl/second. If a flow chamber with different height is used, the preferred flow rates thus have to be adjusted to result in a similar shear rate. Preferably, the shear rate at step d) of a method of the invention is between 1 s⁻¹ and 10000 s⁻¹, more preferably between 10 s⁻¹ and 5000 s⁻¹, more preferably between 50 s⁻¹ and 500 s⁻¹.

In a particularly preferred embodiment the invention provides a method for detecting a cell surface molecule comprising:
a) providing a sensor comprising a sensor surface, said sensor surface comprising a transparent element, preferably a glass prism, covered with a metal film of at most 100 nm thick, preferably a gold or silver film, and at least one binding compound immobilized to said metal film,
b) allowing a liquid cell sample to flow to and along the sensor surface at a flow rate of at least 50 s⁻¹, preferably at least 500 s⁻¹,
c) temporarily reducing shear rate of said liquid sample to allow cells and/or fragments thereof in said liquid sample to bind said binding compound,
d) removing unbound cells or non-specifically bound cells and/or fragments thereof by allowing said liquid sample to flow along the sensor surface at a shear rate of between 1 s⁻¹ and 10000 s⁻¹, more preferably between 10 s⁻¹ and 5000 s⁻¹, more preferably between 50 s⁻¹ and 500 s⁻¹, and
e) determining a presence or absence of a change in refractive index of light incidence at the sensor surface.

Step e) of a method of the invention comprises determining a presence or absence of a change in refractive index of light incidence at the sensor surface. In this step, binding of cell surface molecules to said at least one binding compound is detected by determining a change in refractive index of light incidence at the sensor surface. Preferably, a change in surface plasmon resonance (SPR) angle is determined. A “change in refractive index of light incidence at the sensor surface” as used herein refers to the difference between refractive index of light incidence, preferably SPR angle, measured before a liquid cell sample is allowed to flow to and along the sensor surface and after removing unbound cells or non-specifically bound cells. A change in refractive index and/or SPR angle is indicative of binding of said cell surface molecule to said at least one binding compound, and thus for the presence of cells expressing said cell surface molecule in the analyzed sample. Absence of a change in refractive index and/or SPR angle is indicative of a lack of binding of said cell surface molecule to said at least one binding compound, and thus for the absence of cells expressing said cell surface molecule in the analyzed sample. “Detecting binding” as used herein thus may refer both to detecting whether or not binding has occurred and to detecting the degree of binding. In one embodiment, it is determined whether or not binding of cell surface molecules expressed on cells in the cell sample to a specific binding compound has occurred. This is preferably determined for each spot as element of plurality of spots comprising a single type and specificity of binding compound. If binding to a binding compound specific for a certain cell surface molecule is detected this indicates that the cell surface molecule is expressed on cells present in the cell sample. If binding to a binding compound specific for a certain cell surface molecule is not detected, this indicates that the cell surface molecule is not expressed on cells present in the cell sample. In another embodiment, a method of the invention further comprises determining the relative abundance of cell surface molecules on cells in the liquid sample. As detailed above, the change in SPR angle of reflected light is directly proportional to the binding of analyte on the metal surface of the SPR prism. Thus, the degree of change in SPR angle for a specific binding compound provides information about the amount of cell surface molecules binding to the binding compound and thus about the relative amount of cell surface molecules per cell, i.e. the relative abundance of cell surface molecule, if the concentration of cells in the sample is known. The relative abundance of cell surface molecules on a cell is for instance determined by comparing the degree of binding in two or more cell samples. In that case, it can be established in which sample(s) the relative abundance is higher and in which sample(s) the relative abundance is lower as compared to the other sample(s). Thereby, zygosity (homo- or hetero-zygosity) can be determined, as in homo-zygosity the relative number of cell surface molecules expressed on a cell will higher when compared to hetero-zygosity. Alternatively, the abundance of cell surface molecules on a cell can be determined by comparing the degree of binding in a cell sample with the degree of binding in one or more cell samples of which the relative abundance of cell surface molecules is known. In that case, the relative abundance of cell surface molecules can be quantified. In yet another embodiment, a method of the invention further comprises determining the number of cells in the liquid sample. If the relative abundance of cell surface molecules expressed on a certain cell type is known, the concentration of cells in a sample can be quantified based on the degree of change in SPR angle, and thus the degree of binding to immobilized binding compound.

In step c) of a method of the invention, contact between cells in the liquid sample and binding compound immobilized on the sensor surface, preferably between cell surface molecules and binding compound, is temporarily enhanced. Step c) of a method of the invention comprises reducing the shear rate of said liquid sample to allow cells in said liquid sample to bind said binding compound. Contact between said cells and said binding compound is preferably a result of sedimentation and/or diffusion of cells towards the binding compound. As a result of sedimentation of cells contact between cell surface molecules on these cells and binding compound immobilized on the sensor surface is enabled or enhanced if the liquid cell sample flows over, or is located on top of the sensor surface. Therefore, in a preferred embodiment of the invention, the sensor surface is located below the liquid sample cells in a liquid sample are allowed to sediment onto the sensor surface. Most commercial SPR systems such as BIAcore™ systems (Biacore AB, Uppsala, Sweden), SPRimager systems of GWC Technologies (Madison, Wis., USA), SPRimaging systems of GenOptics (Orsay, France) are configured with optics on top of the fluidics. In such instruments, the liquid, i.e. sample or buffer, flows beneath the sensor surface. Thus, the flow chamber is located underneath the SPR sensor and the incident light is projected from above on the prism and metal film. Such orientation is used to avoid leakage of liquid sample and buffer and of debris into components of the detection system. In these instruments, cell sedimentation thus will occur away from the SPR sensor and will not result in binding of cells to immobilized binding compound. However, because a method of the invention preferably uses sedimentation of cells, the sensor surface is preferably located below said liquid sample. That way, reducing the shear rate of a liquid cell sample to a minimum of the sedimentation velocity of the cells, or preferentially to a complete stop, allows or enhances contact between cells in the sample and the binding compound immobilized on the sensor surface as a result of sedimentation of the cells. SPR imaging systems wherein the SPR sensor and optics are located below the flow chamber and thus below the liquid passing over the sensor are thus particularly suitable for use in a method of the invention. An example of such SPR imaging systems with so-called reversed optics are IBIS-SPR instruments (IBIS Technologies, Enschede, The Netherlands).

The shear rate that allows cells to contact binding compound immobilized on the sensor surface by sedimentation and/or diffusion depends on the cell type present in the liquid sample, in particular on the size of the specific cells. For instance, large cells such as monocytes and several tumour cells will sediment more readily, i.e. at higher sedimentation rate than smaller cells such as red blood cells and platelets. Typically, a reduction of shear rate to less than 10 s⁻¹ will be sufficient to allow all cell types to contact by sedimentation and/or diffusion. Therefore, in a preferred embodiment, shear rate is reduced to less than 10 s⁻¹, more preferably to less than 5 s⁻¹, more preferably to less than 1 s⁻¹, even more preferably to less than 0.1 s⁻¹. Generally, the slower the flow rate, the more readily cells will sediment and the more readily contact between cell surface molecules and binding compounds immobilized on the sensor surface is initiated or enhanced. The best results are obtained when flow is temporarily essentially stopped, because in case of a low shear rate whereby the sedimentation rate is faster than the lateral flow rate, supply of cells to the flow chamber is continued and mainly at the entrance of the flow channel accumulation of cells occurs which is unwanted. As a result an inhomogeneous loading of cells on the sensor surface will occur with a higher density at the entrance of the flow chamber than at the outlet of the flow chamber. Furthermore, essentially stopping the flow rate of the cell sample is preferred because the time required for measurement of a sample is minimized. Therefore, in a particularly preferred embodiment, flow of the liquid sample in step c) is temporarily essentially stopped. This refers to a flow rate of the sample that is essentially zero. “Essentially” is used herein in its general meaning, in that small deviations from zero that do not substantially hamper sedimentation of cells are allowed. If the flow of the liquid sample is essentially stopped, total diffusion of platelets and diffusion and sedimentation of cells such as red blood cells, white blood cells, non solid tumor cells, hybridoma cells, stem or progenitor cells, bacterial cells or fungal cells, occurs.

The period during which the flow rate is reduced, or preferably essentially stopped, depends on the cell type and size of the cells that are analysed and on the type and identity of binding compound immobilized on the sensor surface. For instance, a reduction in shear rate for between 5 seconds and 30 minutes is sufficient to allow sedimentation red blood cells, at the shear rates indicated above. Therefore, in a preferred embodiment wherein the presence of cell surface molecules on red blood cells are determined the shear rate is reduced to between 5 seconds and 30 minutes, more preferably to between 10 seconds and 15 minutes, more preferably to between 20 seconds and 5 minutes, such as for 20, 30, 40, 50, 60, 70, 80, 90, 120, 150, 180, 240, or 300 seconds. More preferably, the flow of the liquid cell sample is essentially stopped during 5 seconds to 20 minutes, more preferably for 10 seconds to 15 minutes, more preferably for 20 seconds to 10 minutes, such as for 20, 30, 40, 50, 60, 70, 80, 90, 120, 150, 180, 240, or 300 seconds.

Further, the height of the flow chamber that contains the liquid cell sample may be adjusted to influence sedimentation of cells. For instance, if a liquid sample with a low concentration of cells is analysed, increasing the height of the flow chamber will increase the number of cells that are sedimented on a particular spot. Following sedimentation, i.e. in step d) of a method of the invention, the height of the flow chamber is preferably reduced again to achieve sufficient velocity and shear rate at a relatively low flow rate. For analysis of a liquid sample having a high concentration of cells a flow chamber of relatively small height may already be sufficient to achieve adequate sedimentation. Therefore, in one embodiment, the height of the flow chamber is adjustable.

In order to measure a low concentration of cells, the flow chamber can be loaded with cells, followed by stopping the flow to sediment, followed by loading again with cells, followed by stopping the flow to sediment. This can be repeated multiple time until a signal of sufficient strength is obtained. Therefore, in one embodiment of the invention, steps b) and c) are repeated at least once following step c) before performing step d) of a method of the invention, such as at least once, at least twice, at least three times, or at least five times Alternatively steps b), c) and d) are repeated at least once, following step d) and before performing step e) of a method of the invention, such as at least once, at least twice, at least three times, or at least five times

In a particularly preferred embodiment the invention provides a method for detecting a cell surface molecule comprising:
a) providing a sensor comprising a sensor surface, said sensor surface comprising a transparent element, preferably a glass prism, covered with a metal film of at most 100 nm thick, preferably a gold or silver film, and at least one binding compound immobilized to said metal film,
b) allowing a liquid cell sample to flow to and along the sensor surface at a flow rate of at least 50 s⁻¹, preferably at least 500 s⁻¹,
c) temporarily reducing shear rate of said liquid sample to less than 10 s⁻¹, preferably to less than 1 s⁻¹, more preferably to less than 0.1 s⁻¹, most preferably temporarily stopping flow of said liquid sample,
d) removing unbound cells or non-specifically bound cells and/or fragments thereof by allowing said liquid sample to flow along the sensor surface at a shear rate of between 1 s⁻¹and 10000 s⁻¹, more preferably between 10 s⁻¹ and 5000 s⁻¹, more preferably between 50 s⁻¹ and 500 s⁻¹, and
e) determining a presence or absence of a change in refractive index of light incidence at the sensor surface.

At least one binding compound is immobilized on the surface of the metal film. The at least one binding compound is specific for a particular cell surface molecule. Suitable binding compounds are antibodies or parts thereof, receptors, proteins, peptides, DARPins, lectins, microorganisms, DNA or RNA molecules or any other biological compound capable of binding a cell surface molecule. With a part of an antibody is meant a part that has the same binding specificity as the antibody. Examples of parts of antibody that are suitable used in accordance with the present invention are a single domain antibody fragments, single chain antibody fragments, single-chain antibody variable fragments (scFvs), Fab fragments, Fab′ fragments and F(ab′)₂ fragments. Suitable proteins for use as binding compound are for instance receptors or cellular ligands such as a. T-cell receptor or Human Leukocyte Antigen (HLA), DARPins (Designed ankyrin repeat proteins), aptamers and lectins. DARPins are a class of non-immunoglobulin proteins exhibiting highly specific and high-affinity target protein binding and are derived from ankyrin proteins consisting typically of three to five repeat motifs of these proteins. DARPins specific for a cell surface molecule of interest can be selected from libraries of DARPins with randomized potential target interaction residues or tailor-made to bind a cell surface molecule of interest. Aptamers are also peptides that are designed to bind specific targets. They consist of a short peptide domain, attached at both ends to a protein scaffold. Lectins are sugar-binding proteins that have high specificity for the respective sugar moiety. Lectins can thus be used as a binding compound for sugar-containing cell surface molecules.

The skilled person will understand that the binding compound may bind indirectly to a particular cell surface molecule. For instance, cell surface molecules can be bound by or attached to a second molecule which is specific for the cell surface molecule and which, in turn, is bound by a binding compound that is specific for the second molecule. Examples of such second molecules are antibodies or parts thereof, receptors, proteins, peptides, DARPins, lectins, microorganisms, DNA or RNA molecules or any other biological compound capable of binding a cell surface molecule. Indirect binding may involve the presence of more than one second molecule located between the cell surface molecule and the binding compound. Suitable binding compounds that indirectly bind a cell surface molecule are the same type of binding compounds described above, i.e. antibodies or parts thereof, receptors, proteins, peptides, DARPins, lectins, microorganisms, DNA or RNA molecules or any other biological compound capable of binding such second molecule. A particularly suitable binding compound that indirectly binds to a cell surface is an antibody or part thereof.

Preferably, a plurality of various binding compounds is immobilized to the metal, preferably gold, film of the sensor surface. This way, expression of a plurality of cell surface molecules in a sample can be analyzed at the same time As detailed herein before, a sensor surface preferably comprises a plurality of spots to which binding compounds can be immobilized. A sensor surface in accordance with the present invention typically comprises 48-96 spots, which means that 48-96 different binding compounds can be immobilized to the metal film of the sensor surface. In one embodiment, the sensor surface further comprises at least one negative control, i.e. a compound that does not specifically bind to any cell surface molecule. For instance, bovine serum albumin (BSA) can be used as a negative control. Alternatively, a plurality of binding compounds can be used in a mixture, whereby multiple binding compounds are immobilized on the sensor surface on each spot. This can for instance be used to immobilize an hybridoma cell fusion mixture on a spot containing anti-CD138 and detect the subsequently produced antibodies by the captured cell by e.g. the immobilized antigen which was used to raise the hybridoma. More ligands can be immobilized together on a spot to allow multi epitope binding of the cell to that particular spot.

Binding compounds are immobilized either directly on the sensor surface, via linking layer, or in a three-dimensional matrix that is in contact with the sensor surface. The latter is used to increase the number of accessible binding compounds to maximize signal. Typically SPR assays use a matrix of dextran molecules on top of the metal surface in which the binding compounds are immobilized, also called a hydrogel. However, cells may not be able to penetrate the dextran layer, which may hamper binding of cells to the immobilized binding compounds. In a method of the invention, for instance a hydrogel of a few nanometers or a linear hydrogel is used so that the hydrogel does not hamper binding of cells to the binding compounds. For direct immobilization on the surface, alkanethiol or disulfide chemistry is for instance used. Further, binding compounds can be immobilized onto the sensor surface via a capture layer which comprises for instance antibodies specific for the binding compound, which antibodies are directly immobilized onto the sensor surface as described.

Also provided is a surface plasmon resonance (SPR) method for detecting a cell surface molecule comprising:
a) providing a sensor comprising a sensor surface, said sensor surface comprising a transparent element covered with a metal film of at most 100 nm thick and at least one binding compound immobilized to said metal film, which binding compound is capable of indirectly binding said cell surface molecule,
b) allowing a liquid cell sample to flow to and along the sensor surface,
c) temporarily reducing shear rate of said liquid sample to allow cells in said liquid sample to bind said binding compound,
d) removing unbound cells or non-specifically bound cells, and
e) determining the ratio of a relative change in refractive index after removing said unbound or non-specifically bound cells and a relative change in refractive index of light after allowing sedimentation and/or binding of said cells to said binding compound, whereby said relative change in refractive index after removing said unbound or non-specifically bound cells is measured after step d) is performed and said relative change in refractive index of light after allowing sedimentation and/or binding of said cells to said binding compound is measured before step d) is performed. Said sensor surface is preferably located below said liquid sample.

Further provided is a surface plasmon resonance (SPR) method for detecting a cell surface molecule comprising:
a) providing a sensor comprising a sensor surface, said sensor surface comprising a transparent element covered with a metal film of at most 100 nm thick and at least one binding compound immobilized to said metal film,
b) allowing a liquid cell sample to flow to and along the sensor surface,
c) temporarily reducing shear rate of said liquid sample to allow cells and/or fragments thereof in said liquid sample to bind said binding compound,
d) removing unbound cells or non-specifically bound cells and/or fragments thereof, and
e) determining the ratio of a relative change in refractive index after removing said unbound or non-specifically bound cells and/or fragments thereof and a relative change in refractive index of light after allowing sedimentation and/or binding of said cells and/or fragments thereof to said binding compound, whereby said relative change in refractive index after removing said unbound or non-specifically bound cells and/or fragments thereof is measured after step d) is performed and said relative change in refractive index of light after allowing sedimentation and/or binding of said cells and/or fragments thereof to said binding compound is measured before step d) is performed.

A “cell sample” as used herein refers to any sample comprising cells or suspected to comprise cells, and/or that comprises cell fragments or is suspected to comprise cell fragments. A cell sample may comprise any type of cell and/or fragments thereof from cell culture or isolated from an organism, such as from an organ, tissue or bodily fluid. Both adherent and non-adherent cells can be analysed with a method according to the invention. As used herein “non-adherent cells” refers to cells that will not adhere to a surface of laboratory equipment when cultured. “Adherent cells” as used herein refers to cells that adhere to a surface of a laboratory equipment when cultured. Adherent cells may be treated to lose their adherent properties before performing a method of the invention, such as by fixation, using for instance paraformaldehyde. Methods for fixating adherent cells are well known in the art. Examples of cells isolated from an organism that can be analyzed with a method of the present invention are cells from blood, saliva, ascites, urine, faeces, skin, muscle, lymph nodes, liver, bone marrow and tumours. Examples of cells from cell culture that can be analyzed with a method of the present invention are hybridoma cells, bacterial cells and fungal cells. A “hybridoma cell” as used herein refers to a cell that is produced from the fusion of an antibody-producing lymphocyte and a non-antibody-producing cancer cell, usually a myeloma or lymphoma and that proliferates and produces a specific monoclonal antibody. Exemplary cell types that can be used in a method in accordance with the invention are red blood cells, white blood cells such as lymphocytes, monocytes, macrophages, neutrophils, eosinophils and basophils, platelets, hybridoma cells, tumor cells, stem or progenitor cells such as hematopoietic stem cells, epithelial cells, endothelial cells, liver cells, intestinal cells. Preferred cell fragments that can be used in a method in accordance with the inventions are microvesicles such as tumor-derived microvesicles. Microvesicles are membrane vesicles of plasma membrane origin that are released by cells into the extracellular environment. They typically range in size from 50 nm to 1000 nm and are shed from cells such as, but not limited to, megakaryocytes, platelets, monocytes, neutrophils and tumor cells. Preferred cell types that are present in a cell sample or suspected to be present in a cell sample are red blood cells, white blood cells, platelets, hybridoma cells, tumor cells, preferably non-solid tumor cells, and stem or progenitor cells.

The cell sample can be used in a method of the invention as it is obtained, or it can be diluted or reconstituted after isolation and/or purification of cells. Suitable buffers for dilution or reconstitution of cells are standard buffers such as PBS buffer, PBST buffer or HBS buffer. Typically, a cell sample used in accordance with the invention comprises between 10⁴ and 10¹⁰ cells/ml. However, the optimal amount of cells used depends on cell type. For instance, about 20×10⁶ RBC and about 10⁹ platelets or microvesicles are typically used.

In one embodiment, the liquid cell sample comprises a crosslinking agent. Such agent crosslinks cells present in the sample which results in amplification of the signal after contact between cells and binding compound is temporarily enhanced. This allows for detection of cells with low cell surface molecule expression, even of cellular antigens that are expressed only in a few copies. A preferred crosslinking agent is high molecular weight dextran, preferably with a molecular weight of more than 5 kD, preferably at least 100 kD, such as around ˜500 kD. The concentration of the crosslinking agent is preferably 1% W/W. As demonstrated in FIG. 6, the weak D red blood cell phenotype could only be detected when 150 kD dextran or 670 kD dextran was present in the sample. The principle of using high molecular weight dextran thus enables discrimination between cells strongly positive for a certain cell surface molecule, cells weakly positive for the cell surface molecule and cells negative for the cell surface molecule. The use of a crosslinking agent such as high molecular weight dextran is particularly suitable for blood group typing. With conventional methods for blood group typing, weak expression of blood group antigen is often not detected and cells are consequently incorrectly typed as negative for this antigen. With a method of the invention, such weak expression can also be detected. Therefore, in a preferred embodiment, a method of the invention comprises blood group typing and the liquid cell sample comprises red blood cells and a crosslinking agent such as high molecular weight dextran of more than 5 kD. More preferably, a method of the invention comprises blood group typing and each liquid sample is analyzed twice, once with addition of a crosslinking agent such as high molecular weight dextran to the sample and once without addition of the crosslinking agent to the sample. This way, strong positive and weak positive expression of blood group antigens can be discriminated.

A method of the invention is preferably performed using an SPR system comprising means for generating back and forth flow. With “back and forth flow” it is meant that a liquid first flows over a sensor surface in a first direction and that subsequently flow is reversed so that the liquid flows in a second direction, which is substantially opposite to first direction. Suitable conditions for back and forth flow fluidics and suitable systems comprising means for back and forth flow are detailed in WO 2012/045325, which is incorporated herein by reference. As a result of back and forth the amount of liquid, sample or buffer, necessary for measurements is relatively small and the time needed for a single measurement is reduced. For instance, back and forth flow fluidics allows analysing samples as small as 70 μl for binding to 96 spots. Another advantage of back and forth flow is that a sample does not need to pass through the pump and/or valves of the liquid transport system thereby avoiding clogging of channels and/or valves, which is particularly a risk when analysing cell samples. In addition, avoiding cell samples to pass through pumps and valves allows for a great increase in the number of measurements that can be performed after regeneration. For instance, Example 1 demonstrates that a single sensor surface with immobilized binding compounds lasts at least 96 cycles of measurement and regeneration. Therefore, in a preferred embodiment, step d) comprises allowing the liquid sample to flow along the sensor surface and the liquid sample in step d) of a method of the invention has a direction of movement opposite to the direction of movement of said liquid sample in step b) of a method of the invention.

In a particularly preferred embodiment the invention provides a method for detecting a cell surface molecule comprising:
a) providing a sensor comprising a sensor surface, said sensor surface comprising a transparent element, preferably a glass prism, covered with a metal film of at most 100 nm thick, preferably a gold or silver film, and at least one binding compound immobilized to said metal film,
b) allowing a liquid cell sample to flow to and along the sensor surface at a flow rate of at least 50 s⁻¹, preferably at least 500 s⁻¹,
c) temporarily reducing shear rate of said liquid sample to less than 10 s⁻¹, preferably to less than 1 s⁻¹, more preferably to less than 0.1 s⁻¹, most preferably temporarily stopping flow of said liquid sample,
d) removing unbound cells or non-specifically bound cells and/or fragments thereof by allowing said liquid sample to flow along the sensor surface at a shear rate of between 1 s⁻¹ and 10000 s⁻¹, more preferably between 10 s⁻¹ and 5000 s⁻¹, more preferably between 50 s⁻¹ and 500 s⁻¹, and
e) determining a presence or absence of a change in refractive index of light incidence at the sensor surface,
   whereby said sensor surface is located below said liquid sample and said liquid sample in step d) has a direction of movement opposite to the direction of movement of said liquid sample in step b).

As described in the Examples, during sedimentation and/or diffusion of cells a non-exponential binding curve is obtained, whereby initially a delay in response is observed after reducing the shear rate or essentially stopping the flow to allow binding between cells and binding compound. Then the response slowly increases in a direct relationship with the number of cells that land on the sensor. The cells that land in a region of interest (RoI) within a spot on the sensor surface, will change the refractive index in the evanescent field, resulting in a positive signal. The signal that is measured after sedimentation and/or diffusion of cells is called the S-response or S-signal (for sedimentation response/signal). The S-signal is thus the relative change in refractive index or SPR angle after sedimentation as compared to baseline or a reference. Following sedimentation and/or diffusion of cells, the flow is increased again thereby washing away unbound or non-specifically bound cells. For reference or control spots, a downward signal is observed, caused by flushing of the sedimented cells. Surprisingly, the present inventors found that for spots with immobilized binding compound that recognizes and specifically binds molecules expressed on the cell surface, an upward signal as compared to the S-signal is observed. The signal that is measured shortly after the flow is increased again is called the T-response or T-signal (for total response/signal). The T-signal is thus the relative change in refractive index or SPR angle after removing unbound or non-specifically bound cells as compared to baseline or a reference. FIG. 1 shows the binding curve that is obtained when cell samples with a method of the invention, and when the S-signal and the T-signal can be determined in a sensorgram.

Thus, if specific binding has occurred, the T-signal after resuming the flow following sedimentation and/or diffusion increases as compared to the S-signal. If no specific binding occurs, the T-signal will not be increased. Without being bound to theory, the surprising increase in T-signal as compared to the S-signal is believed to be caused by an increased pressure on specifically bound cells including a reorientation of the cells towards the sensor surface caused by the resumed flow. As a result, the number of cell surface molecules that comes into contact with immobilized binding compound is increased resulting in an increase in interaction between cells and the sensor surface. Consequently, an increase in signal is observed. Such increase in signal is not detected when cell surface molecule expression is assessed using conventional method whereby cell surface molecules are labeled, such as with fluorescent microscopy.

The present inventors further surprisingly found that the T signal is in direct relationship with the S-signal, i.e. the extent of the signal obtained upon removing unbound or non-specifically bound cells, when flow is resumed after cells are sedimented onto the sensor surface, is in direct relationship with the extent of the signal obtained after sedimentation of cells onto the metal film of the sensor surface. This is most easily explained as a strong S signal is provided by stochastic distribution and sedimentation of cells (see FIG. 1, bottom): the more cells sediment on a surface, the more T signal is observed if the cell contains surface molecules recognized by ligands/receptor on the gold surface. This correlation is demonstrated in FIG. 2, where the T-signal is displayed on the y-axis, and the S-signal on the x-axis. The direct correlation allows for normalization of the data between spots in a single assay by expressing the response as a ratio between both signals, the T/S ratio. This correlation even allows normalization between separate experiments.

Further, it was found that the T/S ratio is a measure for the specificity of binding of cell surface molecules to their respective immobilized binding compound. If the T-signal is higher than the S-signal, this indicates that specific binding between cells and the immobilized binding ligand has occurred. If no specific binding has occurred, resuming the flow will result in washing away essentially all sedimented cells and no increase of the T-signal is observed. Moreover, the higher the T/S ratio, the more specific the binding of cell surface molecules to immobilized binding compounds. As a result, only cells expressing a specific cell surface molecule will give a specific response, i.e. a T/S ratio higher than 1, for a spot on the sensor surface with a binding compound specific for the cell surface molecule. Furthermore, the T/S ratio as a measure for specificity allows that a T/S ratio threshold is set when assessing expression of cell surface molecules, whereby a ratio above the threshold is considered to represent specific binding and thus the presence of the cell surface molecule of interest, and a ratio below the threshold is considered to be an indication of non-specific binding and thus of the absence on the cell surface of the molecule of interest.

In addition to the above, the T/S ratio also provides information about the relative abundance of cell surface molecule expression. The higher the T/S ratio for a specific binding pair, the higher the number of cell surface molecules specifically bound to the immobilized binding compounds, whereby a specific binding pair contains a specific cell surface molecule and binding compound. Thus, when the T/S signal ratio is determined the relative abundance of cell surface molecules can be assessed after comparison with a reference sample containing cells which are known to express a cell surface molecule of interest and, preferably cells of which the abundance of cell surface expression of said molecule of interest is essentially known. Furthermore, as a result of the fact that the T/S ratio is a measure for relative abundance, it can be used to determine homo- or heterozygosity of a cell for a cell surface molecule of interest. A cell that is homozygous for a specific cell surface molecule will have a higher T/S ratio than a cell that is heterozygous for the same cell surface molecule, if the concentration of cells in the analyzed sample is approximately similar.

The skilled person will understand that principle explained above is also applicable to binding compounds that indirectly bind to a particular cell surface molecule and which are specific for a second molecule that is bound or attached to the cell surface molecule.

Thus, now that a method of the invention is provided of detecting cell surface molecules using SPR technology whereby the T/S signal ratio is determined, it has become possible to detect expressed cell surface molecules very accurately, to determine the extent of specificity and to detect the relative abundance of cell surface molecule expression. As detailed above, none of the methods known for determining cell surface molecule expression, allows to determine a quantifiable ratio between signals obtained after sedimentation and/or diffusion and after washing of unbound or non-specifically bound cells. An increase in signal after such washing is not observed. Therefore, no quantifiable ratio, comparable to the T/S ratio found by the present inventors, that is a measure for both specificity and relative abundance of cell surface expression, and in addition can be used for normalization within and between experiments, is available when using such conventional methods.

Accordingly, a preferred embodiment of the invention comprises determining the ratio of a relative change in refractive index or SPR angle after removing said unbound or non-specifically bound cells and a relative change in refractive index of light or SPR angle after allowing binding of said cells to said binding compound. Preferably, the ratio of a relative change in refractive index or SPR angle after removing said unbound or non-specifically bound cells and a relative change in refractive index of light or SPR angle after sedimentation of cells is determined. The relative change in refractive index or SPR angle after sedimentation and/or binding of said cells to said binding compound is called the S-signal or S-response. The S-signal is preferably measured just before unbound or non-specifically bound cells are removed in step d), e.g. 1-50 seconds, preferably 1-30 seconds, more preferably about 15 seconds, before step d) of a method of the invention is performed. Said flow is preferably in a direction opposite to the direction of flow in step b). The relative change in refractive index or SPR angle after removing said unbound or non-specifically bound cells is called the T (total) signal. The T-signal is preferably measured just after resuming and/or increasing the flow in step d) of a method of the invention, e.g. 1-50 second, preferably 1-30 seconds, more preferably about 15 seconds, after resuming and/or increasing flow. Thus, in a preferred embodiment, the T/S ratio for each spot on the sensor face is determined. “Relative” as used herein refers to a change in refractive index or SPR angle from a baseline or reference signal. A baseline or reference signal as used herein refers to a signal from a reference spot, such as a change in refractive index or SPR angle of a liquid sample not comprising cells, of a liquid cell sample from a spot on the sensor surface without immobilized binding compound or of a liquid cell sample before sedimentation. Preferably said baseline signal is obtained using a liquid cell sample whereby the sensor surface does not contain immobilized binding compounds, for instance by lacking immobilized compound or containing a compound that does not specifically bind to cell surface molecules such as BSA.

Provided is thus a method for detecting a cell surface molecule comprising:
a) providing a sensor comprising a sensor surface, said sensor surface comprising a transparent element covered with a metal film of at most 100 nm thick and at least one binding compound immobilized to said metal film,
b) allowing a liquid cell sample to flow to and along the sensor surface,
c) temporarily reducing shear rate of said liquid sample to allow cells and/or fragments thereof in said liquid sample to bind said binding compound,
d) removing unbound cells or non-specifically bound cells and/or fragments thereof, and
e) determining the ratio of a relative change in refractive index after removing said unbound or non-specifically bound cells and a relative change in refractive index of light after allowing binding of said cells to said binding compound.

Preferably, the ratio of a relative change in refractive index after removing said unbound or non-specifically bound cells and a relative change in refractive index of light after sedimentation of cells and/or binding of said cells to said binding compound is determined. Furthermore, the relative change in refractive index is preferably measured 1-50 seconds, preferably about 15 seconds, before step d) of a method of the invention is performed and 1-50 second, preferably about 15 seconds, after resuming and/or increasing flow in step d) of a method of the invention.

When using the cell sedimentation technique as described herein, it has to be noted that cells will settle down onto the sensing surface non-specifically. This means that the SPR sensorgram will show a non-specific binding-like event which could indicate binding to the sensor surface. This increase in signal however is not (by default) related to interaction of cell surface receptors on the cells to the specific ligands but the increase is due to the gradual sedimentation of cells penetrating the evanescent field on top of the sensing surface. The sensorgram shows a typical increase in response only seen in cell based sedimentation protocols which is characterized by a “fishhook” shaped curve, though this fishhook curve is a typical feature of cell based SPR measurements it becomes more profound if there are sample differences when comparing to for instance the system buffer. One example would be temperature, if the sample has a considerably different temperature (in our case 15 degrees Celsius) when comparing to the system buffer (37 degrees Celsius) then the sensorgram will show this by a more obvious fishhook curve. The curve however can be corrected by subtraction to a certain extend when one chooses to reference (subtract) the data with a reference area that is not showing any ligand specific binding. After injecting cells in the flow chamber an initial delay in the response occurs. The delay can be explained by the fact that the flow is switched off after the cell sample is injected into the microfluidic chamber and the cells are allowed to sediment. While injecting the cells in the fluidic chamber a laminar flow profile exists and the cells are forced to flow in the middle of the flow chamber without touching the walls. The largest velocity gradient exists close to the wall where a so called depletion layer of cells is formed. When the flow is stopped some time is needed in order for the cells to sediment into the evanescent field from which point on they are detected as a refractive index shift by the IBIS MX96 instrument. The time it takes for the cells to sediment into the evanescent field is dependent on the type of cell, particularly the weight of the cells or specific density of the cells with respect to the buffer and the injection velocity which creates the depth of the depletion layer, but generally it is observed that it is between 30-60 seconds for the cells and the injection velocity (80 μl/second) that were applied in the experiments. Cells will sediment non-specifically on the whole sensor surface area. However, the specificity of a cell measurement occurs already in the sedimentation stage, or in SPR terms association stage [x] where a steep increase in signal is detected on top of the collective sedimentation signal. The cells that attach to their respective specific ligands are pulled much stronger towards the surface as opposed to the same cell lines that are allowed to sediment and settle on top of a surface that was not coated with a specific ligand (reference area). As the cells which interact specifically with the ligands are gradually binding to the surface, the cells are assumed to start to spread themselves and as such are allowing more ligands to interact with their cell surface, causing the cells to get pulled into the evanescent field even more thus making them more clear to see and causing a higher sensorgram and non-stable (drifting) response when comparing to a non-specific interaction. In FIG. 11A after the sedimentation phase the drift of the signal caused by cell action e.g. spreading or non-specific anchoring is an important aspect. The cells that are not binding with the immobilized ligands remain relatively superficial on the sensing surface but can be distinguished from specifically bound cells as they have not initiated extensive binding or flattening (yet) as the specifically bound cells, this phenomenon causes them to penetrate the evanescent field more shallowly and as such generate “weaker” signals as opposed to specific cells. The adherent process did not yet start yet and the cells can be removed after limited time.

Another important phenomenon to take into account when doing cell based SPR is that there is a distinct difference between adherent and non-adherent cells (f.i. skin cells and blood cells respectively). The main characteristics that become apparent when comparing the 2 types of cells is that the adherent cells start after a period of time to show non-specific binding which is no longer able to be washed off by the system buffer without adding any regenerative additives to it or without taking special chip inactivation precautions (BSA coating). Non-adherent cells do not show this kind of behavior and as the results show will be washed off even after a longer amount of time, so long as they are not specifically bound to a ligand.

The non-specific adherent cell behavior can be disabled to a certain extent, enabling distinction of specific and non specific binding, by using EDTA as an additive to the sample buffer. EDTA blocks function of pathways (such as the cadherin pathway) which are involved in the mechanics behind the adherent behavior of adherent cells. The addition of EDTA prevents the non specific binding by working as a chelating agent. The cells in such a PBS-EDTA buffer then will only bind specifically to an immobilized ligand if they express the corresponding antigen on the cell membrane. So using the PBS-EDTA buffer will ensure that the binding signals that are seen in the SPR sensorgram are not attributable to the adherent behavior of certain cell lines, but is more likely due to an interaction of the immobilized ligands and the antigen on the cell membrane. Using BSA in a coupling buffer as a chip deactivation agent additionally enhances the prevention of non-specific binding to a certain extent.

The signal strength that a cell based sample can produce is dependent on the number of cells in the sample (and as such the number of cells that sediment on the chip), however it is also dependent on the use size of the RoI. By changing the sizes of the RoI's the amount of RU of the binding events can thus be increased or decreased. The smaller the RoI the higher the potential response will be from a binding event (f.i. antibody-antigen interaction, cell sedimentation etc.), although it has to be noted that background noise also increases. On the other hand making the RoI bigger will decrease the background noise, but will also make potential responses from interactions smaller. It is therefore imperative to find a balance between the size of the RoI and the amount of background noise/specific RU response.

In one embodiment, a method of the invention further comprises adding a second binding compound competing with said at least one binding compound for binding to the cell surface molecule to said liquid cell sample. “Competing with” as used herein means that the second binding compound has the same specificity as the immobilized binding compound. As used herein, “the same specificity” means that the second binding compound is specific for the same cell surface molecule as the immobilized binding compound immobilized and preferably also for the same epitope of the cell surface molecule. The degree of specificity is not necessarily the same. If binding of cell surface molecules is abrogated after addition of the second binding compound, this indicates that binding to the immobilized surface molecule was indeed specific. One embodiment of the invention therefore comprises removing cell surface molecules bound to the binding compound immobilized on the sensor surface by addition of a second binding compound having the same specificity as said binding compound immobilized on the sensor surface. Further, this competition principle can be used to analyze the presence of binding compounds such as antibodies in a second sample. For instance, if a liquid cell sample comprising red blood cells or white blood cells is analyzed and the binding compound is an antibody specific for blood group antigens or serotype specific antigens, respectively, a second sample that is added can be tested for the presence of allo-antibodies or auto-antibodies. If binding of cell surface molecules in the first sample is reduced, the second sample comprises a binding compound that competes with the binding compound immobilized to the sensor surface.

In another embodiment, a method of the invention further comprises disrupting cells bound to said at least one binding compound and adding at least a second binding compound to said disrupted cells or parts thereof bound to said at least one binding compound. As used herein “disrupting” cells refers to any process that results in release of the contents of the cell due to a loss of cell integrity. Examples of such process include physical method such as applying high shear forces in high flow regimes, and chemical methods, such as lysing cells using well known lysis buffers or permeabilizing cells using well known permeabilization buffers. With such method, cells that specifically bind to the immobilized binding compound through their cell surface molecules following sedimentation of the cells, are disrupted from the sensor surface either physically, such as by high shear forces in high flow regimes, or chemically using cell lesion buffers. This way, cells are disrupted whereby parts of the cell, e.g. (parts of) cell membranes, comprising the bound cell surface molecule remain bound to the sensor surface via the immobilized binding compound but parts of the cell lacking the cell surface marker are washed away. Alternatively, cells are permeabilized allowing said second binding compound to bind to intracellular antigens. This is followed by injection of binding compounds which bind to antigen expressed on the remaining membranes. Subsequently, the presence or absence of intracellular membrane bound antigen is detected by determining a presence or absence of a change in refractive index of light incidence at the sensor surface. Such method is particularly suitable for determining the presence or absence of membrane bound antigens that are located intracellularly and exposed and accessible for binding to specific binding compounds after disruption of the cell. An example of such antigens are G protein coupled receptors (GPCRs), of which the intracellular part can be accessed with a method of the invention. This way, a single SPR assay can be used for detection of both cell surface molecules expressed on a cell and intracellular membrane bound antigens. Said second binding compound is thus preferably capable of specifically binding a intracellular membrane bound antigen, whereby “specifically binding” has the meaning as described herein before. Disrupting cells bound to said at least one binding compound is also useful to obtain a stable signal because the signal of living cells that are assessed for their cell surface expressed molecules may vary during the life of the cell due to changes in binding to the immobilized binding compounds. After disruption of the cell, a stable signal will be obtained because changes in binding can no longer occur.

The invention further provides a dual assay method wherein at least one antibody in the liquid cell sample is detected, whereby at least one binding compound is specific for a cell surface molecule and at least one binding compound is an antigen. Preferably, a plurality of binding compounds specific for a cell surface molecule and a plurality of antigens are immobilized to the metal film of the sensor surface. Said immobilized binding compounds are capable of binding cell surface molecules expressed on cells in the liquid sample and said antigens can be recognized by antibodies or parts thereof that may be present in the liquid sample. This way, the presence of both cell surface molecules expressed on cells and antibodies present in the sample can be determined simultaneously in a single sample. Such method is for instance useful for auto- or allo-antibody screening in a patient sample together with determining cell surface molecule expression. This is for instance particularly suitable for determining the presence of both blood group antigens expressed on cells and (auto- or allo-) antibodies against blood group antigens in a single assay using a, optionally diluted, blood sample comprising red blood cells. With such method, two non-coupled measurements can be done using one sample injection consisting at least cells and analytes (e.g. antibodies). Determining the presence or absence of one or more antibodies or parts thereof in the sample is preferably performed prior to determining the presence or absence of one or more cell surface molecules. Thus, determining the presence or absence of one or more antibodies or parts thereof comprises determining a presence or absence of a change in refractive index of light incidence or change in SPR angle at the sensor surface prior to step c) of a method of the invention. The presence or absence of one or more antibodies or parts thereof is for instance determined during step b) of a method of the invention. Measurement of the binding of antibodies to immobilized antigens at particular spots preferably comprises measuring the slope of the signal per second, i.e. dR/dt. Following such measurement, the shear rate is temporarily reduced or flow is essentially stopped to allow sedimentation and/or diffusion of cells in the liquid sample and resumed to remove unbound or non-specifically bound cells, thereby preferably determining the T/S signal ratio of the S-response and T-response of the binding of cell surface molecules expressed on cells to immobilized binding compounds at particular spots as detailed above. One embodiment of the invention therefore comprising further comprising detecting at least one antibody in said sample, whereby at least one binding compound is specific for a cell surface molecule and at least one binding compound is an antigen, whereby a change in refractive index due to binding of said at least one antibody to a blood group antigen is measured before sedimentation, i.e. before step c), and a change in refractive index due to binding of said at least one cell surface molecule to said antibody or a part thereof, or protein specific for a blood group antigen is measured after sedimentation but before removing unbound or non-specifically bound cells and after removing said unbound or non-specifically bound cells, i.e. at least one measurement between steps c) and d) and at least one measurement after step d). Preferably, back and forth flow is used to prevent detection of cell binding before binding of antibodies in the liquid sample is completed. Sedimentation of cells to the sensor surface during measurement of antibody binding interferes with determining the dR/dt for antibody binding. Such dual assay method according to the invention enable determining both the presence of a plurality of antibodies in a sample and the present of a plurality of cell surface markers in a single assay very fast, i.e. within 3-5 minutes.

With a method of the invention information about the identity of cell surface molecules, the relative abundance thereof, and the concentration of cells expressing specific surface molecules can be obtained. As a result, a method of the invention can be used for a wide variety of applications. For instance, many diseases are characterized by expression of specific cell surface antigens, by an increase in expression of cell surface antigens, or by an increase or decrease in specific cell types in a particular organ or tissue. For example, several tumours can be diagnosed on the basis of expression of tumor-specific antigens. As another example, the amount of white blood cells and the relative concentration of white blood cell types may be an indicator or disease or of disease state. Yet another example of a use of a method of the invention is serotyping to determine the most appropriate donor-recipient match for organ transplants.

A method of the invention is particularly suitable for blood group typing. Blood group typing is for instance performed for establishing compatibility between blood donors and recipients. Cell surface antigen determination methods such as flow cytometry and fluorescent microscopy typically allow for four to six antigens to be simultaneously tested routinely, a number too low for detailed antigen screening of red blood cells (RBC). Therefore, the inexpensive agglutination-based methods are still being used for RBC typing to assure safe blood-matching between donor and transfusion recipient. Currently, all blood donors in for instance the Netherlands are serologically typed for the AB0, DCcEe and K antigens. A subpopulation is also typed for other clinically relevant antigens (e.g. FY, JK, MNS) to be able to provide fresh antigen-negative blood for individuals that have developed antibodies against those antigens. Both direct and indirect agglutination immunoassays are well known in the art. In these assays, the agglutination of RBC expressing blood group antigen to which antibody is bound is used to indicate the presence or absence of the corresponding antigen.

Agglutination-based detection in cartridges or in tubes for RBC-typing, is cheap but laborious and not suitable for multiplexing. Further, such tests may lack the required sensitivity for the detection of small amounts of antigen and are prone to subjectivity in the interpretation of results.

The present inventors found that an SPR method as provided herein allows for fast, sensitive and label-free blood group typing. A method of the invention allows for simultaneous testing of a plurality of blood group antigens. The number of antigens that can be measured simultaneously is only limited by the number of spots on the sensor surface. For instance, a sensor surface with 96 spots allows for the measuring of 96 antigens at the same time. Further, with a method of the invention, a single measurement can be performed in just minutes. Another advantage of a method of the invention is that detection of blood group antigens is very sensitive. Even weak signals can be picked up which possibly would have given a negative result in conventional blood group typing methods. Before the present invention, no fast, sensitive and accurate method of simultaneous and label-free typing of blood group antigens was available. The invention thus provides an economical high-throughput method for RBC typing and antibody screening which reduces the hands-on time and costs, provide automation, and to increase donor-recipient matching in the general blood bank practice. Therefore, in a preferred embodiment, the cell surface molecule is a blood group antigen, and said at least on binding compound is specific for a blood group antigen. Particularly, the invention provides a method for blood group typing comprising:
a) providing a sensor comprising a sensor surface, said sensor surface comprising a transparent element, preferably a prism, covered with a metal film of at most 100 nm thick, preferably a gold or silver film, and at least one binding compound specific for a blood group antigen immobilized to said metal film,
b) allowing a liquid cell sample comprising red blood cells to flow to and along the sensor surface,
c) temporarily reducing shear rate of said liquid sample to allow sedimentation of red blood cells in said liquid sample, preferably essentially stopping the flow of said liquid sample,
d) removing unbound cells or non-specifically bound red blood cells, and
e) typing blood group antigens by determining a presence or absence of a change in refractive index of light incidence at the sensor surface. Preferably, step e) comprises detecting the presence or absence of binding of blood group antigens to said at least one binding compound is determined by determining a change in refractive index of light incidence at the sensor surface.

Suitable binding compounds immobilized to the metal film of a sensor surface for use in blood group typing are antibodies or parts thereof specific for blood group antigens, or proteins, such as receptors or cellular ligands, e.g. a T-cell receptor or Human Leukocyte Antigen (HLA), lectins, aptamers and DARPins, capable of specifically binding a blood group antigen. The development of specific antibodies, aptamers and DARPins is well known in the art. Lectins can be used as a binding compound for sugar-containing blood group antigen, such as antigens A and B. Up to now, over 600 different blood group antigens have been identified and the list of blood group antigens is still growing. Examples of most common blood group systems are the AB0 blood group system, Rhesus blood group system, MNS blood group system, Kell blood group system, Lewis blood group system, P blood group system, Lutheran blood group system, Duffy blood group system, Kidd blood group system and other blood group system/antigens including Diego, Yt, XG, Scianna, Dombrock, Colton, Landsteiner-Wiener, Chido/Rodgers, IIh/Bombay, Kx, Gerbich, Cromer, Knops, Indian, Ok, Raph, JMH, Ii, Globoside, GIL, Rh-associated glycoprotein, Vel, Er, Batty, Lan, Ata, Jra, AnWj, Sda, Biles (Bi), Box (Bxa), Christiansen, HJK, HOFM, JFV, JONES, Jensen, Katagiri, Livesay, Milne, Oldeide, Peters, Rasmussen, Reid, REIT, SARA, Torkildsen, Bg. Therefore, in a preferred embodiment, an antibody or a part thereof, or a protein immobilized on the metal film of a sensor surface is specific for a blood group antigen of the AB0 blood group system, i.e. antigen A and/or B, Rhesus blood group system, i.e. RhD, RhC, Rhc, RhE, Rhe, MNS blood group system, Kell blood group system, Lewis blood group system, P blood group system, Lutheran blood group system, Duffy blood group system, Kidd blood group system and/or a blood group system or antigen selected from the group consisting of Diego, Yt, XG, Scianna, Dombrock, Colton, Landsteiner-Wiener, Chido/Rodgers, Hh/Bombay, Kx, Gerbich, Cromer, Knops, Indian, Ok, Raph, JMH, Ii, Globoside, GIL, Rh-associated glycoprotein, Vel, Er, Batty, Lan, Ata, Jra, AnWj, Sda, Biles (Bi), Box (Bxa), Christiansen, IIJK, HOFM, JFV, JONES, Jensen, Katagiri, Livesay, Milne, Oldeide, Peters, Rasmussen, Reid, REIT, SARA, Torkildsen, Bg.

In a preferred embodiment, the invention therefore provides a method for blood group typing comprising:
a) providing a sensor comprising a sensor surface, said sensor surface comprising a transparent element, preferably a prism, covered with a metal film of at most 100 nm thick, preferably a gold or silver film, and a plurality of binding compounds selected from the group consisting of antibodies specific for a blood group antigen and proteins, such as a receptor or cellular ligand, e.g. a T-cell receptor or Human Leukocyte Antigen (HLA), DARPins, aptamers and lectins, capable of specifically binding a blood group antigen, immobilized to said metal film,
b) allowing a liquid cell sample comprising red blood cells to flow to and along the sensor surface,
c) temporarily reducing shear rate of said liquid sample to allow sedimentation of red blood cells in said liquid sample, preferably essentially stopping the flow of said liquid sample,
d) removing unbound cells or non-specifically bound red blood cells by allowing said liquid sample to flow along the sensor surface, and
e) typing blood group antigens by determining a presence or absence of a change in refractive index of light incidence at the sensor surface,
   whereby said sensor surface is located below said liquid sample and preferably wherein said liquid sample in step d) has a direction of movement opposite to the direction of movement of said liquid sample in step b). Step d) preferably comprises determining the ratio of a relative change in refractive index after removing said unbound or non-specifically bound cells and a relative change in refractive index of light after allowing binding of said cells to said binding compound.

The invention further provides a method wherein at least one binding compound is an antibody or a part thereof, or a protein specific for a blood group antigen and at least one binding compound is a blood group antigen. Preferably, a plurality of antibodies or parts thereof, and/or proteins specific for a blood group antigen and a plurality of blood group antigens are immobilized to the metal film of the sensor surface. This way, the presence of both blood group antigens expressed on cells and antibodies against blood group antigens can be determined simultaneously in a single sample. Blood group antigens expressed on cells will bind to the immobilized antibody or part, or protein specific for this antigen, and antigen-specific antibodies will bind to immobilized blood group antigens. Such method further allows for screening for the presence of allotype- and/or antigen-specific antibodies (e.g. anti-A/anti-B/anti-RhD) generated in a patient either naturally or after pregnancy or previous blood transfusion. This can be done using either infrequent typing RBC expressing all most common antigens in a heterozygous state (“full house”), or by utilizing in vitro generated RBC expressing all common antigens by expansion of genetically manipulated RBC progenitor- or stem cells. These RBC will bind all antigen-specific spots on an SPR sensor surface. However, if serum of the patient containing antibodies are added to the sample containing RBC expressing all common antigens, these RBC will be released from specific spots containing a specific blood group binding compound (e.g. antibody) if the serum contains antibodies corresponding to the specificity of the binding compound spotted on the chip. This way, the presence and identity of allotype- and/or antigen-specific antibodies present in the serum of the patient can be determined. Preferably, a method of the invention further comprising detecting at least one antibody in said sample, whereby at least one binding compound is an antibody or a part thereof, or protein specific for a blood group antigen and at least one binding compound is a blood group antigen, comprises measuring a change in refractive index before binding of cells to said antibody or a part thereof, or protein specific for a blood group antigen, i.e. between steps b) and c), and measuring a change in refractive index after binding of said cells to said antibody or a part thereof, or protein specific for a blood group antigen but before removing unbound or non-specifically bound cells and after removing said unbound or non-specifically bound cells, i.e. at least one measurement between steps c) and d) and at least one measurement following step d). With said measurement before binding of cells to said antibody or a part thereof, or protein specific for a blood group antigen, a change in refractive index resulting from binding of antibody in the liquid sample to immobilized a blood group antigen is detected. With said measurements after binding of cells to said antibody or a part thereof, or protein specific for a blood group antigen and after removing unbound or non-specifically bound cells, a change in refractive index resulting from binding of cells, or more specifically cell surface molecules, to said immobilized antibody or a part thereof, or protein specific for a blood group antigen is detected. As described herein before, determining the presence or absence of one or more antibodies against blood group antigens is preferably performed prior to determining the presence or absence of one or more blood group antigens. Measurement of the binding of antibodies to immobilized blood group antigens preferably comprises measuring the slope of the signal per second, i.e. dR/dt. Following such measurement, the flow rate is temporarily reduced to allow sedimentation of cells in the liquid sample and resumed to remove unbound or non-specifically bound cells, thereby preferably determining the T/S signal ratio of the S-response and T-response of the binding of blood group antigens expressed on red blood cells to immobilized binding compounds as detailed above.

As detailed herein before, a binding compounds may indirectly bind to a cell surface molecule. Such method of the invention is particularly suitable to detect and/or quantify opsonized cells, such as antibody- or Ig-opsonized cells. Opsonization refers to the binding of an opsonin, such as an antibody, to an antigen present on a cell or pathogen. In this way a cell or pathogen is marked for phagocytosis. The Fab portion of the antibody binds to the antigen of the cell or pathogen, and the Fc portion of the antibody binds to an Fc receptor on the phagocyte. This principle can be used in accordance with the present invention when the antibody that binds to an antigen is subsequently bound by a binding compound immobilized to an SPR sensor surface. Examples of antibody-opsonized cells that can be detected and/or quantified with a method of the invention include opsonized red blood cells, white blood cells such as lymphocytes, monocytes, macrophages, neutrophils, eosinophils and basophils, platelets, microvesicles, hybridoma cells, tumor cells, stem or progenitor cells such as hematopoietic stem cells, epithelial cells, endothelial cells, liver cells, intestinal cells. Antibody-opsonized cells can be detected using an array of antibodies recognizing all Ig isotypes and subclasses as binding compound immobolized to the SPR sensor surface. Such application is for instance useful in the diagnosis of autoimmune diseases that are characterized by opsonisation of cells by auto-antibodies. For instance, a method of the invention can be used to detect and quantify auto-antibodies in thrombocytopenias, such as idiopathic thrombocytopenic purpura (ITP), which may be characterized by the presence of auto-antibody-opsonized platelets. In such application, binding compounds can be immobilized to the SPR sensor surface that are specific for Ig isotypes and subclasses. As demonstrated in Example 6, with such method even very low numbers of opsonized platelets can be detected.

The invention therefore provides an SPR method for detecting and/or quantifying Ig-opsonized platelets comprising:
a) providing a sensor comprising a sensor surface, said sensor surface comprising a transparent element, preferably a prism, covered with a metal film of at most 100 nm thick, preferably a gold or silver film, and a plurality of binding compounds selected from the group consisting of Ig isotype and Ig subclass specific antibodies, immobilized to said metal film,
b) allowing a liquid platelet sample comprising platelets to flow to and along the sensor surface,
c) temporarily reducing shear rate of said liquid sample to allow sedimentation of platelets in said liquid sample, preferably essentially stopping the flow of said liquid sample,
d) removing unbound platelets or non-specifically bound platelets by allowing said liquid sample to flow along the sensor surface, and
e) detecting and/or quantifying the presence of opsonized platelets by determining a presence or absence of a change in refractive index of light incidence at the sensor surface,
   whereby said sensor surface is located below said liquid sample and preferably wherein said liquid sample in step d) has a direction of movement opposite to the direction of movement of said liquid sample in step b). Step d) preferably comprises determining the ratio of a relative change in refractive index after removing said unbound or non-specifically bound platelets and a relative change in refractive index of light after allowing binding of said platelets to said binding compound. Such method is suitably used for diagnosis of thrombocytopenia.

The invention further provides a surface plasmon resonance (SPR) sensor comprising a transparent element covered with a metal film of at most 100 nm thick, said sensor surface comprising a plurality of spots, each spot comprising at least one binding compound immobilized to said sensor surface, preferably to the metal film, wherein said sensor surface comprises a plurality of binding compounds selected from the group consisting of antibodies specific for a blood group antigen and proteins, such as DARPins and lectins, capable of binding specifically binding a blood group antigen. Such SPR sensor is particularly suitable for blood group typing in accordance with the invention. Said transparent element is preferably a prism. Said metal is preferably gold or silver, most preferably gold, and said metal film is preferably between 20 and 80 nm thick, more preferably between 40 and 60 nm, most preferably between 45 and 55 nm Said sensor preferably comprises at least 4 binding compounds selected from the group consisting of antibodies specific for a blood group antigen and/or proteins, such as DARPins, aptamers and lectins, capable of binding specifically binding a blood group antigen, more preferably at least 5, such as 6, 7, or 8 blood group antigens. Said antibodies or parts, or protein are preferably selected from the group consisting of antibodies or parts, or protein specifically binding antigens of the AB0 blood group system, i.e. antigen A and/or B, Rhesus blood group system, i.e. RhD, RhC, Rhc, RhE, Rhe, MNS blood group system, Kell blood group system, Lewis blood group system, P blood group system, Lutheran blood group system, Duffy blood group system, Kidd blood group system and/or a blood group system or an antigen selected from the group consisting of Diego, Yt, XG, Scianna, Dombrock, Colton, Landsteiner-Wiener, Chido/Rodgers, Hh/Bombay, Kx, Gerbich, Cromer, Knops, Indian, Ok, Raph, JMH, Ii, Globoside, GIL, Rh-associated glycoprotein, Vel, Er, Batty, Lan, Ata, Jra, AnWj, Sda, Biles (Bi), Box (Bxa), Christiansen, HJK, HOFM, JFV, JONES, Jensen, Katagiri, Livesay, Milne, Oldeide, Peters, Rasmussen, Reid, REIT, SARA, Torkildsen, Bg. Most preferably, at least the antigens of the AB0 system, RhD, RhC, Rhc,RhE, Rhe and K are immobilized on the sensor surface.

The invention further provides an SPR sensor according to the invention further comprising red blood cells. Said red blood cells are preferably bound to the binding compounds selected from the group consisting of antibodies specific for a blood group antigen and proteins, such as DARPins and lectins, capable of binding specifically binding a blood group antigen immobilized on the sensor surface.

In one embodiment, the SPR sensor surface further comprises a plurality of blood group antigens. Such SPR sensor is particularly suitable for blood group typing in accordance with the invention whereby both blood group antigens expressed on cells and antibodies against blood group antigens are analyzed. Said sensor surface preferably comprises at least 4 blood group antigens, more preferably at least 6, more preferably at least 8, more preferably at least 10 blood group antigens. Said blood group antigens are preferably selected from the group consisting of antigens of the AB0 blood group system, Rhesus blood group system, MNS blood group system, Kell blood group system, Lewis blood group system, P blood group system, Lutheran s blood group system, Duffy blood group system, the Kidd blood group system, or an antigen selected from the group consisting of Diego, Yt, XG, Scianna, Dombrock, Colton, Landsteiner-Wiener, Chido/Rodgers, Hh/Bombay, Kx, Gerbich, Cromer, Knops, Indian, Ok, Raph, JMH, Ii, Globoside, GIL, Rh-associated glycoprotein, Vel, Er, Batty, Lan, Ata, Jra, AnWj, Sda, Biles (Bi), Box (Bxa), Christiansen, HJK, HOFM, JFV, JONES, Jensen, Katagiri, Livesay, Milne, Oldeide, Peters, Rasmussen, Reid, REIT, SARA, Torkildsen, Bg. In an examplary embodiment, at least 2 antigens of the AB0 blood group system, 4 antigens of the MNS system, the P antigen, 2 antigens of the Lutherian system, 2 antigens of the Kell system, 2 antigens of the Lewis system, 2 antigen of the Duffy system, and 2 antigens of the Kidd system, and the Rhesus blood group system are immobilized on the sensor surface.

The invention further provides a surface plasmon resonance (SPR) measuring system comprising an SPR sensor according to the invention.

The invention is further explained in the following examples. These examples do not limit the scope of the invention, but merely serve to clarify the invention.

### BRIEF DESCRIPTION OF THE DRAWINGS

FIG. 1. Cellular detection using multiplex SPR. Sensorgram showing binding of RBC to 3 specific spots with anti-M and three reference spots without ligands. After stopping the flow, the cells sediment on the sensor surface non-specifically and a sedimentation signal (S) was observed for all regions of interests. After restarting the flow, washing unbound cells, an upward signal (T) was measured for cells that bind specifically to selected spots, while control spots return to baseline level. Top insert is an SPR image (reflectivity) of the sensor surface showing 3 specific anti-M spots with RBC, and 9 negative spots are visible, while the spotted protein is visible on two spots (middle row, second from left, bottom row, second from right). Bottom insert is a microscopic view of a single spot with 4 quadrants of RoIs showing M±RBC bound to an anti-M spot. The SPR response is a measure of the amount of RBC per Region of interest. The resonance unit (RU) shift per RoI was independent of the size of the RoI's in a homogeneous sedimented cell population. RU corresponds with 10⁻⁶ times the refractive index unit (RIU). E.g. water has ˜RIU=1.33. So shifts should be measured in the sixth decimal. A RoI area of 45×45 pixels corresponds to ˜250×250 gm and acquired 200 RBC resulting in ˜8000 RU response, or ˜40 RU/RBC. However, a saturation effect was observed for higher numbers of cells per surface area (data not shown).

FIG. 2: Typing of RBC on a SPR-biosensor. Gold sensors spotted with anti-M, anti-RhD, anti-Glycophorin A, or human serum albumin were used to type RBC (typed with standard agglutination methods) and the signals measured as explained in FIG. 1. A) The total response (T) on anti-M spots was plotted against the sedimentation response (S), resulting in a highly significant correlation (p<0.0001) for M+ cells, but not for M− cells. The response (T/S) results therefore in a normalized response value of the typed RBC. B) Accordingly, anti-M, anti-D, anti-Glycophorin A (positive control), and HSA (negative control) spots can be used to reliably type the antigen-make up of RBC. In the figure the S-line has the normalized value of 1 for correcting/normalizing the differences in the sedimentation signal. A value higher than 1 means that after sedimentation an upward signal is measured. The absolute response per surface area correlates to the number of cells per RoI (FIG. 1). We therefore expressed the response on each spot for each cell as T/S.

FIG. 3: On-chip anti-RBC screening. A. Schematic representation of antibody screening on Chip. B. Gold sensors spotted as in FIG. 2, were injected with anti-M antibodies after the dissociation phase (FIG. 1) and washing. This treatment has no affect for spots with RBC bound to other antigens (e.g. Anti-D), but RBC bound to anti-M spots gradually lost binding and where washed away.

FIG. 4: Repeated accurate detection. Robust detection and accurate typing of red blood cells for at least ˜100 times on a single chip. Six examples of specific RBC antigen typing, of which anti-N is least specific, but still allows for discrimination between N-positive and negative cells. In this experiments, Red blood cells that were either positive and negative for a given antigen, except for Lutheran B, where no negative cells were available as they are very uncommon, were alternatively injected for 96 times and the T/S response recorded.

FIG. 5: Amplifying signals with Dextran. As the expression of some antigens can be low, for example on “weak D” cells, we induced crosslinking of the RBC by carrying the experiment out as in FIG. 2, but in the presence or absence of increasingly larger dextran. For some antigens like RhD it results in amplification of the specific signals, while for others it has no effect (anti-M). The numbers in the X axis legends refer to molecular weight of Dextran

FIG. 6: Weak D signals can be picked up. The presence of dextran increased the signal strength (T/S) and allowed for detection of “weak D” cells. At the same time, it increased the signal of normal RhD-expressing cells. Weak RhD (RhD positive but weakly expressing the protein, two donors shown) RBC and their signals on a anti-RhD, or blanco or HSA control spots. Weakly expressing cells are correctly diagnosed as RhD+ and can be discriminated from RhD− cells.

FIG. 7. Combined RBC antigen typing and antibody screening in one experiment. The principal of first detecting antibodies, then cellular antigens was tested by spotting BSA (control), anti-IgG, and M on different spots. A mixture of IgG and RBC was flowed over the chip, first in back and forth motion, allowing for specific-detection of IgG, then stopping the flow allowing for sedimentation of RBC on all spots, then starting the flow allowing for specific-detection of the M antigen.

FIG. 8. Hybridoma cells. Other cells, in this case non-adherant VU1D9 hybridoma (not capable of spreading) behave more similarly as RBC, non-CD138 expressing cells do not bind, but CD138-VU1D9 do and result in a specific detection on CD 138 spots.

FIG. 9: Platelets. Although much smaller and less dense than RBC, platelets can also detected with this technology. After injection of platelets and stopping the flow, platelets do not sediment but apparently enter the stagnant layer by random movement after stopping the flow, only sticking to spots with specific ligands (anti-human platelet antigen Ia, IIPA1a, antibodies), allowing their specific detection. This does not occur on control IgG spots or non-spotted spots, where the platelets apparently bounce back outside the evanescent field. This in agreement with the observation that isolated platelets, unlike other cells like RBC in solution, do not sediment. When the flow starts, this results in an increase in the T/S response, allowing for even more specific detection.

FIG. 10: Tumour cells. Sensorgram of adhering cells SKBR3 on G-type chips. Tumour cells (EpCAM—expressing breast cancer cell line SKBR3) behave intermediately to RBC and platelets, sedimenting after stopping the flow, resulting in increasing adherence to spots if they contain a ligand (anti-EpCAM) compared to EpCAM-negative HS578T ductal breast carcinoma cells. Starting the flow results in a upward response which is in contrary to the non-adherent (e.g. RBC) cells that stabilize in flow. Non-epcam expressing cells adhere and probably spread non-specifically on EpCAM spots and less at HSA spots. EpCAM expressing cells allow for discrimination between tumour phenotypes.

FIG. 11. Cell measurement characteristics of adherent cells compared to non-adherent cells. A. Lower line: Non-adherent cells bind specifically to immobilized ligands and after starting the flow a stable T/S signal is measured. Upper line: Adherent Epcam positive cells get pulled more to the surface gradually as they bind to more ligands and start to spread out resulting in drift of the T/S signal. B. Signals of multiple spots after sedimentation of adherent cells.

FIG. 12. Schematic representation of cell binding to SPR chip. Representation of cell spreading on an SPR chip during a measurement. 1. The flow chamber is still empty as the cell sample is being injected. 2. Cells sediment and gradually fall into the evanescent field. 3. Cells sedimented fully and attach to their specific ligands. 4. Under the influence of ligand binding, cells start to spread out and get pulled deeper into the evanescent field causing a stronger increase in SPR signals for specifically bound cells as opposed to non-specifically bound cells.

FIG. 13. Quantifying auto- or allo-antibodies in thrombocytopenia. A) Control platelets were isolated and run over a IBIS chip containing C17 (recognizing platelet gpIIbIIIa as positive control), IgG isotype (negative) control, and anti-Immunoglobulin isotypes (IgG, IgA, and IgM) plus control spots (blanco, HSA). B) same as in A, but the platelets were now opsonized with recombinant anti-HPA1a antibody (B2G1) recognizing the HPA1a epitope on GPIIbIIIa. C) Platelets from a patient with thrombocytopenia. In time, positive control, but not negative controls result in deposition of platelets onto the spots (in quadruplo, depicted as means and standard deviations). D) At the end of the run, the response at 1200 seconds were plotted with standard deviations. Patients X responses were significantly higher than background for IgG and IgM, not IgA.

### EXAMPLES

### Example 1

### Red Blood Cells

As a proof of principle for a multiplex red blood cell (RBC) typing on a SPR imaging device, a planar carboxy-modified gold layer sensor without hydrogel (IBIS Technologies, Enschede, The Netherlands) was spotted with IgG monoclonal antibodies specific for RBC-antigens using a Continuous Flow Microspotter (CFM, Wasatch Microfluidics (Salt Lake City, Utah, US) (Lokate, A. M. C., et al. 2007. J. Am. Chem. Soc. 129, 14013-14018). In total 48 spots were created with ligands of anti-RBC antigen immunoglobulins including anti-M (mouse IgG1, LM140/110 Merck/Millipore, Scotland, GB), anti-D (human IgG1, 8D8, Sanquin, Amsterdam, The Netherlands), anti-glycophorin A (mouse IgG1, CLB-ery-1, AME1, Sanquin) and reference spots with human serum albumin (HSA, Sigma-Aldrich A8763) and phosphate buffered saline (PBS). Anti-M recognizes the M-antigen of the MNS blood group system. Anti-D reacts with the Rhesus-D antigen present on D+ RBCs, and absent on D− RBCs. Anti-glycophorin A is directed against glycophorin A, the most abundant membrane glycoprotein of the RBC, and the carrier protein of the MNS allo-antigen system. The monoclonals were purified by Protein-A (GE Healthcare) and rebuffered to a PBS buffer. An optimal coupling concentration and pII for immobilization of antibodies and IISA was found to be around 2 μg/ml in 10 mM acetate at pH 5.3 based on spotting density and responses to antigen-positive versus negative cells (data not shown). After the spotting process, the sensor was positioned in the IBIS MX96 (IBIS Technologies, Enschede, Netherlands). Samples were exposed to all spots at the same time and interactions to all 48-spots were monitored in real-time The sensor surface can be used many times by a regeneration process where the RBC are removed from the covalently coupled ligands keeping assay costs low. Detailed operation of the predecessor of the IBIS MX96 instrument and the CFM are described elsewhere (Lokate, A. M. C., et al. 2007. J. Am. Chem. Soc. 129, 14013-14018 and Natarajan S. et al. 2008. Anal. Biochem. 373, 2008 141-146).

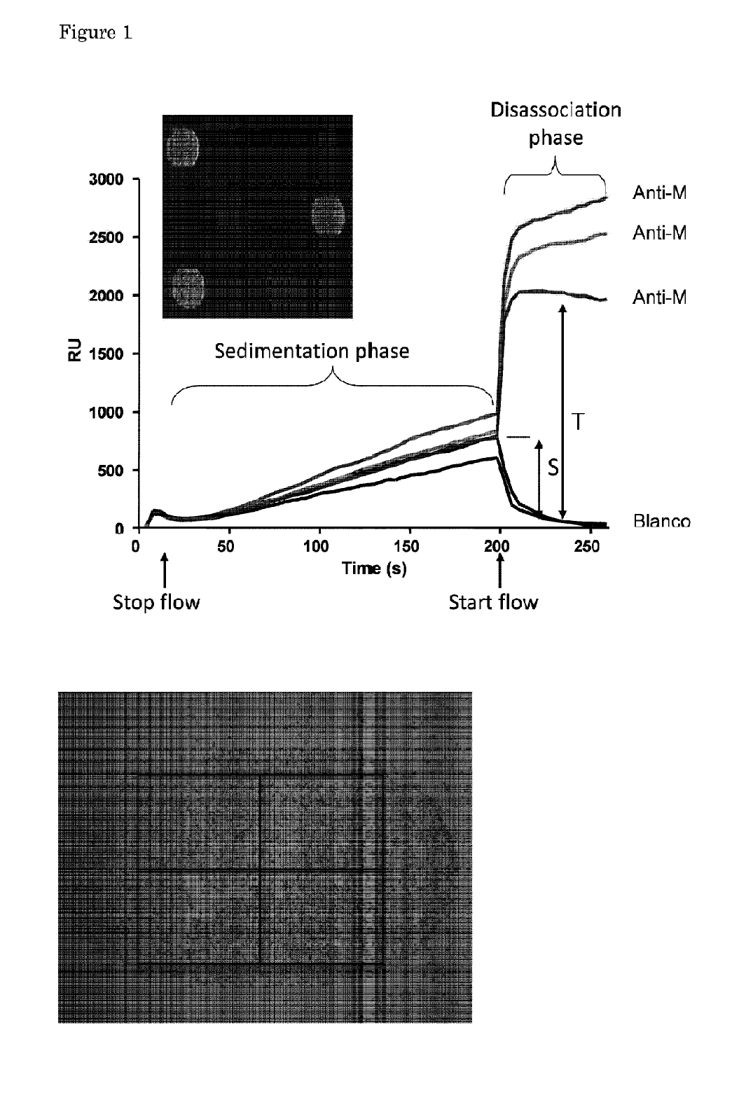

Prior to injection of the cells, the diluted RBC suspension (20*10⁶ cells/ml, obtained from 5 μl blood) was both mixed (by repeated aspirating and dispensing) and transported from a microtiter plate (200 μl) using the built-in autosampler by XYZ positioning of the needle via Teflon tubing into the flow chamber. Mixing was necessary to homogenize a pellet of cells in the microtiter plate well. The flow chamber has dimensions 6 (w)×10 (l)×0.2 (h) mm. A total volume of 800 μl was aspirated into the tubing and a cell suspension of 200 μl was passed into and through the flow chamber with a flow speed of 120 μl/s. After stopping the flow for two minutes after 40 seconds a slow accelerated increase of response is observed while RBC sediment onto the complete sensor surface, including the reference spots. However, after resuming the flow after 2 minutes of sedimentation at 20 μl/s in the backward direction, unbound cells and superfluous cells were washed from the surface (FIG. 1).

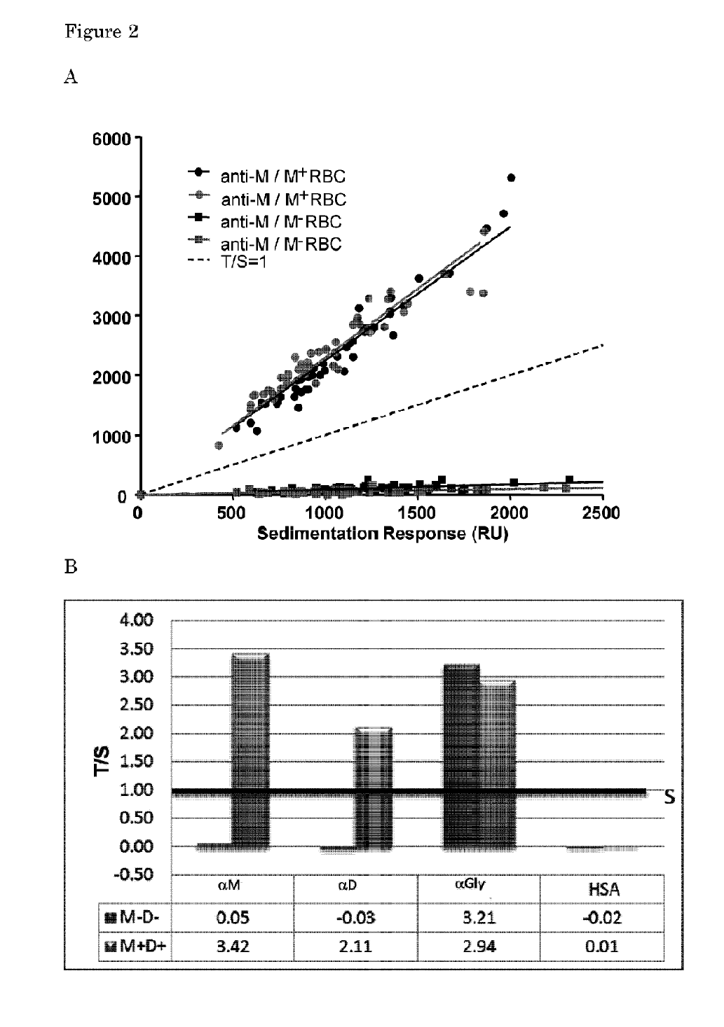

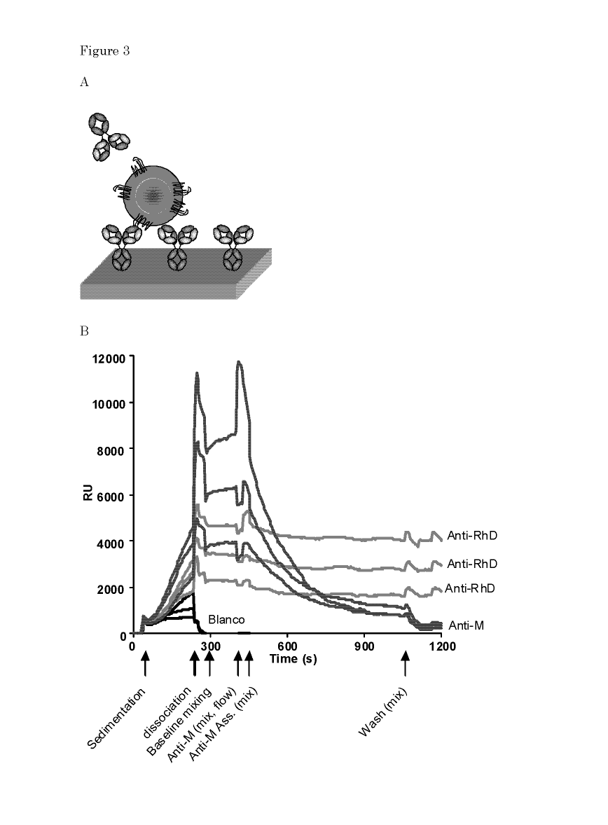

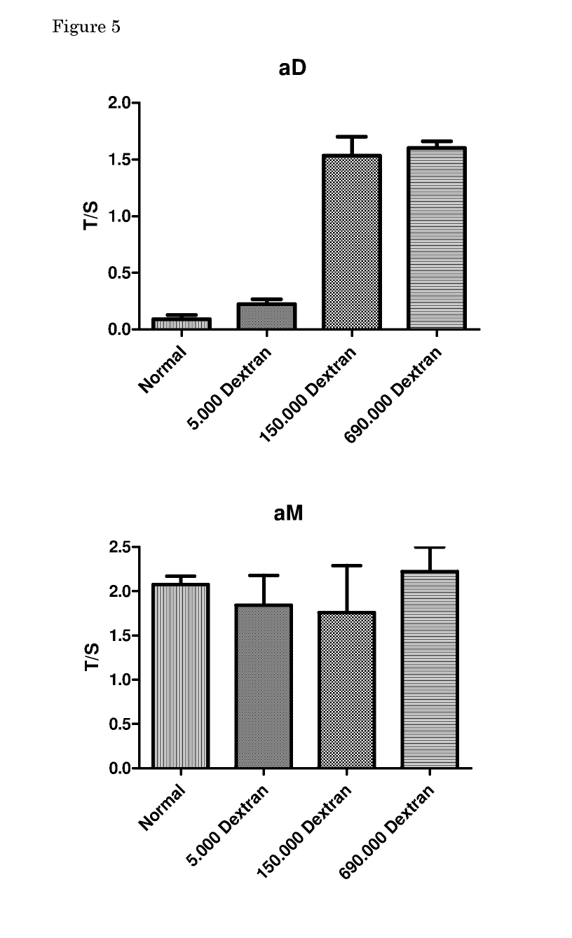

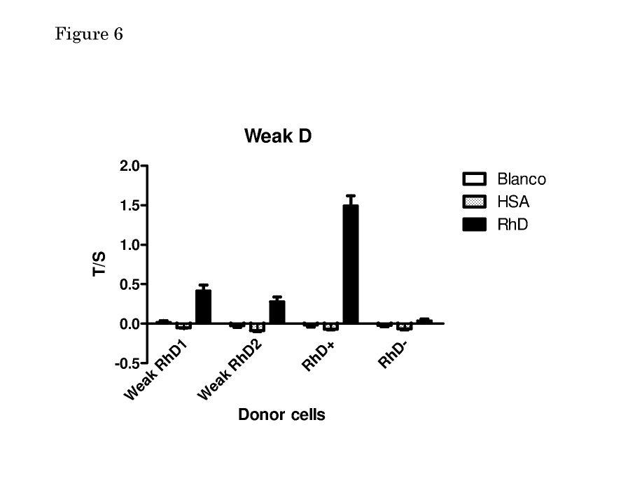

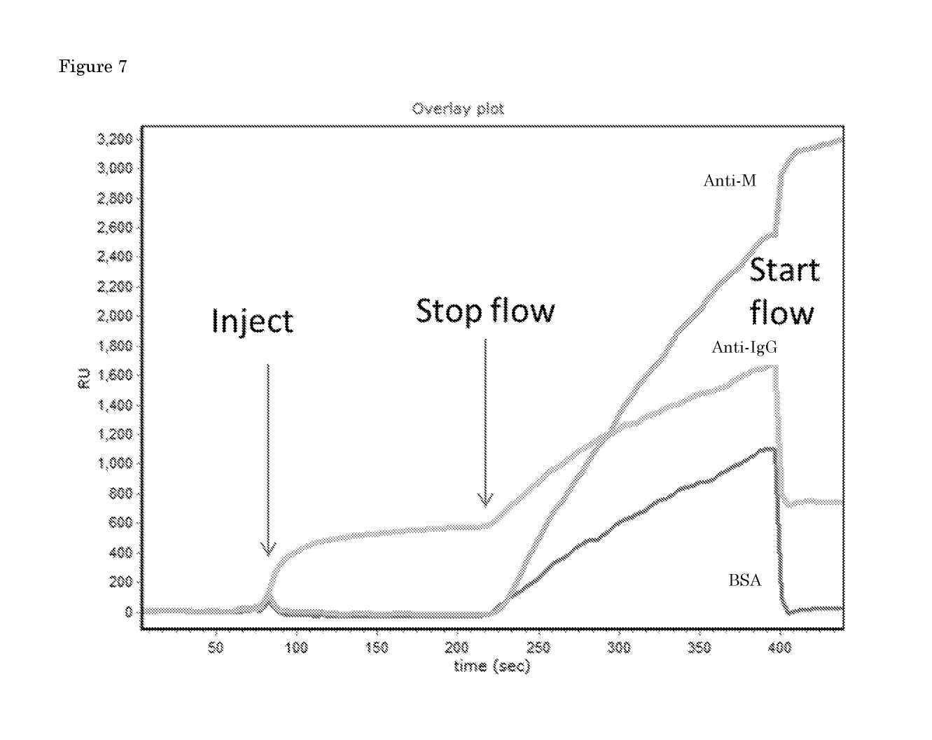

While the kinetic curves during sedimentation show a non-exponential binding curve, a delay in response is always observed after stopping the flow. Then the response slowly increased in direct relationship with the number of cells that land on the surface (data not shown). The cells that land in a region of interest (RoI, user determined within spots, FIG. 1 inlay) will change the refractive index in the evanescent field accordingly, resulting in a positive signal. For all spots a so-called S-response can be measured (FIG. 1). After resuming the flow, washing away unbound cells, a downward signal to the baseline was observed on reference spots, or if the cell was negative for antigen-specific spot, caused by flushing of the sedimented cells. However an upward signal was observed for spots with antibodies recognizing antigens on the cell surface (FIG. 1). For some spots the upward signal (R) can be three times the S-response >15000 RU. We observed some unequal distribution of RBC over the whole chip, with some spots having less RBC sediment and therefore lower S signal. Importantly, the magnitude of the specific total response (T, FIG. 1) was in direct relationship with the sedimentation response (FIG. 2A), allowing for normalization of the data between spots and between experiments by expressing the specific response as a ration between T/S (FIG. 2B). Only cells gave a specific response of the correct phenotype, allowing for accurate detection of the expressed blood group (FIG. 1B). This signal could also be amplified using dextran sedimentation, allowing for detection of cells with low antigen expression, as is the case for the weak D phenotype, that can be discriminated from D-negative cells after sedimentation with high molecular weight dextran (FIGS. 5 and 6).

To prove the specificity of the interaction on the chip, we injected antibodies over the cells bound to the chip, and observed that the bound RBC lost their interaction with the chip if the injected antibodies were of the same specificity as on the spots (e.g. anti-M releases RBC bound to M-spots, FIG. 3).

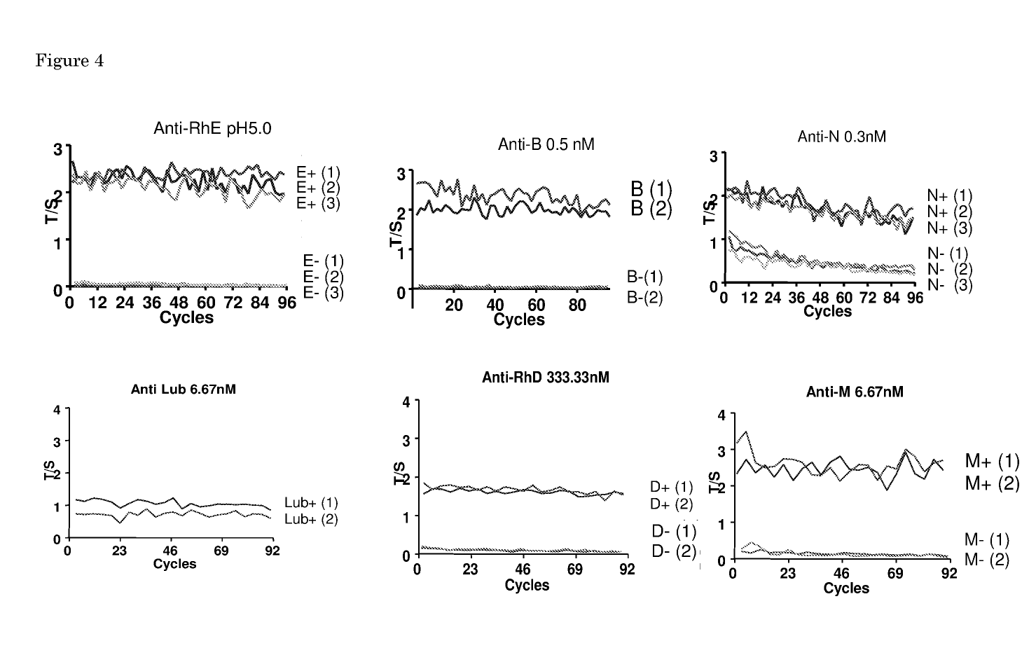

Robust detection and accurate typing of red blood cells was assessed by repeated detection for at least ˜100 times on a single chip (FIG. 4). In this experiment, red blood cells that were either positive and negative for a given antigen, were alternatively injected for 96 times and the T/S response recorded. FIG. 4 demonstrates that accurate detection can be performed at least 96 times on the same chip.

From these observations the kinetic process of cell binding has at least four typical features:
First, after injecting the cells, a delay of response signal is observed after stopping the flow. This typical delay is only observed when large particles (cells) are applied but never when e.g. an antibody sample is injected.
Second, cells that bind specifically to the immobilized ligand molecules will show an upward response, after starting the flow again, while a downward response is observed from reference spots or other spots that do not bind cells. Positive cells displaying a upward response, give a downward response, approaching the sedimentation response once the flow is stopped again (FIG. 3). If the flow rate is below a certain critical value of disruption, the response is stable and the cells will stay on the spots and no off-rate is observed.
Third, the interaction-strength of the RBC with the surface can be increased by crosslinking the cells, e.g. with crosslinking agents like high-molecular weight dextran for RBC, increasing the avidity, allowing for detection of cellular antigens expressed only in few copies.
Fourth, the interaction on the chip can be abrogated by injecting a competing agents like antibodies of the same specificity, thereby verifying the specificity of the interaction. This principle may therefore also be used to screen for the presence of allo-, even also auto-antibodies to a given antigen.

These typical effects can be explained as follows:
1. While cells are flowing in tubing and in flow chambers, the laminar flow will deplete the stagnant layer of cells close to the wall. In other words after a lateral transport of the cell suspension over the sensor surface, cells are not in direct contact with surfaces, but will stay in the middle of the stream. Therefore the red blood cells will first enter the cell-free stagnant layer before settling in the evanescent field of the sensor and hence the delay of sedimentation-response is observed.
2. When cells are bound to specific spots of the sensor surface, the flow will induce a reorientation, repacking, or pressing down of the biconcave RBC to the surface. Energetically it is more beneficial that cells are pressed closer to the surface induced by the flow. This results in a larger part of the specifically bound cells that is localized in the evanescent field and an upward response is observed. In contrast, sedimented cells which are not recognized by the coated antibodies (and by reference spots) will be washed away and the response will return to the baseline. However when the flow is too high (data not shown), the shear forces to the cells will increase too and the cells will disrupt from the surface. Hence, discrimination can be made between weak/non-binding cells compared to adhering cells by adjusting the flow speed in the backward direction after the sedimentation phase, and by the use of cellular crosslinkers.

This technology can therefore be used for extensive patient/donor matching by:
A. Typing the antigen profile of RBC by applying the patient's and donor's RBC over an anti-RBC antigen spotted chip. To be able to detect low antigen expression (e.g. “weak D”), high molecular weight (e.g. 690 kD) can be used to crosslink the RBC, thereby increasing their avidity to the spots.
B. Screening for the presence of allotype/antigen-specific antibodies (e.g. anti-A/anti-B/anti-RhD etc) generated in the patient either naturally or after pregnancy or previous transfusion. This can be done using either infrequent typing RBC expressing all most common antigens in a heterozygous state (“full house”), or by utilizing in vitro generated RBC by expansion of genetically manipulated RBC progenitor- or stem cells. These cells will bind all antigen-specific spots, but will be released if serum contains antibodies corresponding to the specificity of the antibodies spotted on the chip.

### Example 2

### Combination of Cell Surface Molecule Screening and Blood Group Antigen Typing in Red Blood Cells

Experiments were carried out as in FIG. 1, but first the presence of IgG in addition to RBC antigens was measured. BSA (control), anti-IgG, and M were coupled to different spots. A mixture of IgG and RBC was flowed over the chip, first in back and forth motion, allowing for specific-detection of IgG, then stopping the flow allowing for sedimentation of RBC on all spots, then starting the flow allowing for specific-detection of the M antigen. Note that after resuming the flow, the binding to the anti-M spot is increased, while it is reduced back to baseline on the BSA and anti-IgG spots. For the anti-IgG spot, the baseline is equal to the response measured by specific interaction of IgG with the anti-IgG spot before stopping the flow. Thus, the presence of for example anti-RBC (e.g. anti-A, anti-B) in a sample can be performed simultaneously with RBC typing.

### Example 3

### Hybridoma Cells

Experiments with hybridoma cells were performed as described in Example 1.

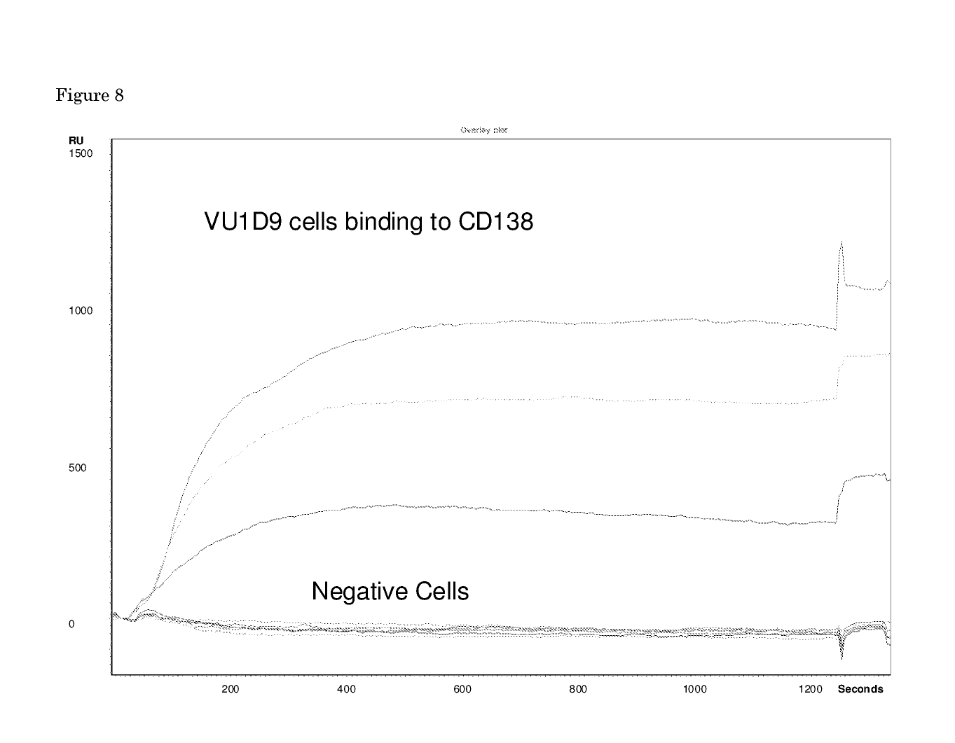

Hybridoma cells are non-adherent cells. Non-specifically bound cells can easily be washed away, allowing for highly specific signal (FIG. 8). This also indicates that non-adherent tumour cells, e.g. from blood, can also be detected and easily discriminated from non-tumour marker-bearing cells in this way.

### Example 4

### Platelets

Experiments with platelets were performed as described in Example 1. The platelet suspension was passed into and through the flow chamber with a flow speed of 150 μl/s. After stopping the flow for two minutes, flow was resumed at 20 μl/s in the backward direction.

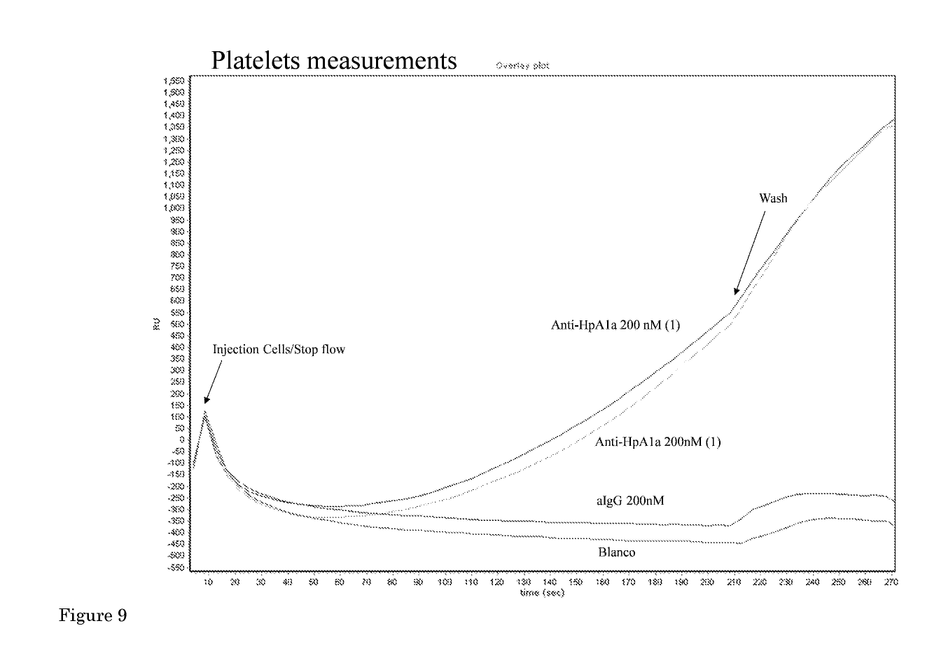

Unlike RBC, the platelets do not sediment—not in solution—nor is this observed on the spotted gold chip. However, the platelets do enter the evanescent field when the flow is stopped, only to escape (or bounce back) rapidly and are therefore not detected (control IgG spots FIG. 9), unless specific anti-platelet antibodies (e.g. against Human Platelet Antigen 1, HPA1) are present on the spot (FIG. 9. Anti-HPA1a spots). Those platelets are retained on the spot, resulting in a positive response measured on the chip. Once the flow is resumed, this results in a slight and temporary (until 30 min. data not shown) elevation of the response, similar to what is observed with RBC, probably because more cell volume is pushed into the stagnant layer, and therefore into the evanescent field, resulting in an increased signal.

### Example 5

### Adherent Tumour Cells

In this example the SPR response when cells with different EpCAM antigen densities are exposed to EpCAM antibody coated sensor surfaces is evaluated.

Materials and Methods

SPR

The MultipleX 96 (MX96, Ibis technologies by, Enschede, the Netherlands) was used for SPRi measurements. The IBIS MX96 has the capacity to measure 96 parameters simultaneously in a single measurement and uses back and forth flow minimizing the amount of sample and reagents needed for a measurement.

CFM Spotter

For spotting ligands on the gold sensor surfaces the Continuous Flow Microfluidic (CFM) spotter was used (Wasatch microfluidics LLC, Salt Lake City, Utah, USA). The CFM spotter has the ability to spot up to 48 different ligands onto the sensor chip simultaneously under back and forth confined flow. In two spotting sessions 96 ligands can be printed onto a single sensor. The confined back and forth flow increases the efficiency of the spotting and avoids the risk of evaporation for contact and non-contact droplet based spotting methods.

SPR Chips

Easy2Spot G-type chips were used (SSens by, Enschede, the Netherlands) as gold SPR sensor surfaces. The chips have a dextran gel layer, which enables a more accurate replication of freely moving ligands. The sensors are delivered pre-activated.

Antibodies

Anti EpCAM antibody was generated using the VU1D9 hybridoma. Goat anti mouse IgG F(c) antibody was acquired from Rockland immunochemicals inc, Gilbertville, Pasadena, United States of America.

Cells

Cells from the breast cancer cell lines HS578T, MCF7 and SKBR3 cells were used for the SPRi measurements. The cells were harvested using a common trypsinisation protocol after which they were resuspended in culture medium, washed with PBS and finally resuspended in PBS-EDTA 0.25% and used at a concentration of ˜2 million cells per ml. The EpCAM density of these cell lines was determined by flowcytometry (ref) for SKBR3 cells.

Chip Deactivation Agent

A 1% Bovine Serum Albumin solution (BSA) (Sigma-Aldrich chemie GmbH, Steinheim, Germany) in sodium acetate immobilization buffer was used as a deactivation agent. A stock solution of 2-Aminoethanol (MP Biomedicals LLC, Illkrich, France) was used to create a 100 mM 2-Aminoethanol solution with a pH of 8.

Ligand Immobilization Buffer

A 10 mM solution of immobilization buffer at pH 4.5 was made using anhydrous sodium acetate (Sigma-Aldrich chemie GmbH, Steinheim, Germany) and acetic acid (Merck Schuchardt OHG, Hohenbrunn, Germany). First a 0.2 M stock solution was made of both components, then from these stock solutions 1.93 parts of sodium acetate were mixed with 3.07 parts of acetic acid, finally 95 parts of ultrapure demineralized water were added. The pH was checked and if needed adjusted to reach 4.5.

System Buffer

PBS 10× was created in house according to common protocol. EDTA di sodium salt was acquired from VWR. To the 1× PBS system buffer of the IBIS MX96 0.0003% Tween 20 (Thermo Fischer Scientific PLC, Waltham, Mass., USA) and EDTA (Sigma) at a concentration of 0.25% was added to reduce the adherence of cells to the chips.

Cell Binding and Cell Adherence

To run cell samples on the MX96 system a custom script for cell handling was developed. Cells were prepared at a concentration of ˜2×10⁶/ml in PBS. A 600 μl cell sample and a blank containing 600 μl of PBS buffer were pipetted into 600 μl PCR vials and placed into the sample rack of the MX96. The experiment was then set up to run the samples under identical conditions.

A G-type chip was spotted with goat anti mouse anti IgG F(c) antibody at a concentration of 20 μg/ml and anti EpCAM with an identical concentration. The antibodies were immobilized in the CFM spotter using sodium acetate for 60 minutes. As negative controls for the spotting process sodium acetate buffer and 0.51% BSA solutions were used. For chip deactivation 100 mM 2-amino-ethanol was flown over the chip after loading the sensor into the MX96. Deactivation time was 10 minutes.

For SPRi measurements forty-eight regions of interest (roi's) were placed over the chip. In total 20 roi's were analysed of which 10 were spotted with goat anti mouse anti IgG F(c) antibody at a concentration of 20 μg/ml and the other 10 were spotted with directly immobilized anti-EpCAM antibody. Just before aspiration of the cells, the samples were first resuspended automatically by the MX96 to prevent cell clumping while awaiting aspiration. An entire run comprised of an EpCAM capturing phase for 20 minutes (to obtain anti EpCAM capturing on the anti IgG spots), an association phase (called sedimentation phase when no flow is applied), followed by a dissociation phase in which PBS is passed for 30 minutes under defined flow conditions and a regeneration phase in which 1M Glycine-HCL pH 2 is passed for 60 seconds.

Cell Binding with Varying Ligand Concentrations.

A G-type chip was spotted with a serial dilution of goat anti mouse anti IgG F(c) antibody at an initial concentration of 20 μg/ml and with a serial dilution of anti EpCAM at an identical initial concentration. The antibodies were immobilized in the CFM spotter using sodium acetate for 60 minutes. As negative controls for the spotting process sodium acetate buffer and 1% BSA solutions were used. Before each sample EpCAM antibodies were injected at 10 for 20 minutes to obtain anti EpCAM capturing on the anti IgG spots. The cells were then injected and allowed to associate for 30 minutes followed by a dissociation phase for 30 minutes. At the end of each run, regeneration with pH 2 Glycine HCl was performed for one minute.

Chip Optimization

To reduce non-specific sticking of cells to the SPR sensor the chip, the chip deactivation protocol after ligand immobilization in the CFM spotter was changed. A 1% BSA solution in sodium acetate buffer was used as a deactivation agent for 15 minutes prior to using 100 mM 2-Aminoethanol also for 15 minutes. This was done to ensure total chip inactivation of any possible “open” chip areas that were not spotted with ligands.

TABLE 1
Sample series that was used for the comparison of specific and non-
specific binding using cells. First anti EpCAM was captured, then
the cells were flown over the sensing surface and lastly there was
a regeneration intended to reset the chip to its initial state.

| Sample | | | |
|---|---|---|---|
| number | Capture | Sample | Regeneration |
| 1 | Anti EpCAM | PBS (Blank) | pH 2 Glycine |
| | 10 μg/ml | | HCl |
| 2 | Anti EpCAM | HS578T in | pH 2 Glycine |
| | 100 μg/ml | PBS EDTA 0.25% | HCl |
| 3 | Anti EpCAM | SKBR3 cells in | pH 2 Glycine |
| | 10 μg/ml | PBS EDTA 0.25% | HCl |
| 4 | Anti EpCAM | MCF7 cells in | pH 2 Glycine |
| | 10 μg/ml | PBS EDTA 0.25% | HCl |

Results

SPRi Measurements of Cells with Antibodies Couples to the Chip Surface by Different Means and Concentrations

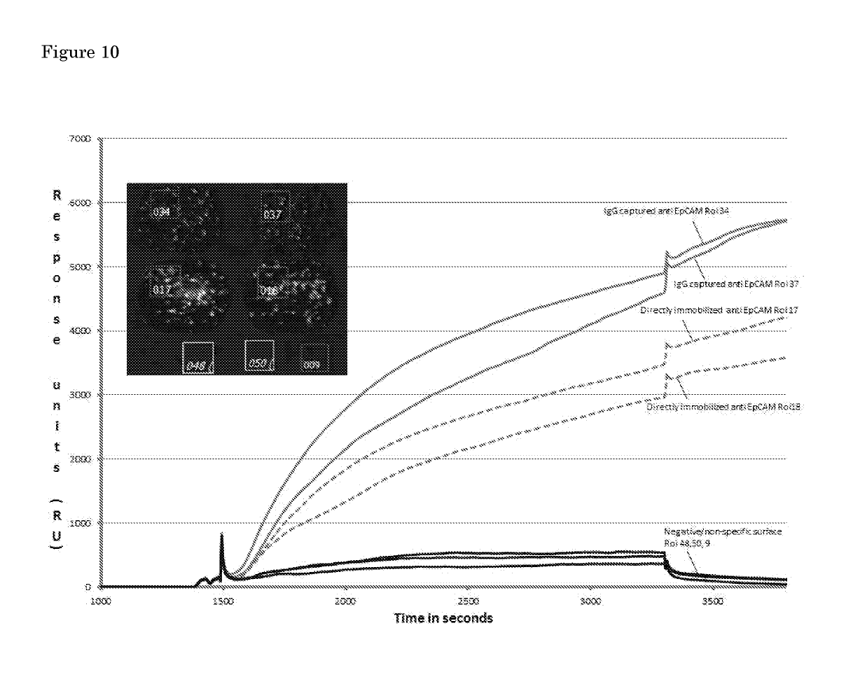

FIG. 10 shows a sensorgram of binding to selected spots with SKBR3 cells. After flow across the chip has been initiated at 80 μl/sec the cell suspension is injected as visualized by the first spike in the SPR response. There is a considerable stronger signal on the anti EpCAM spots as opposed to the anti EpCAM negative spots. Additionally the anti EpCAM spots on which the antibody was captured using an anti IgG antibody are considerably higher in response compared to the directly immobilized anti EpCAM. The slope of the response appears higher with increasing concentration of antibody on the spots (not shown here). After 30 minutes the flow is restarted as can be observed by the second spike in the response curve. The response curves of the anti EpCAM spots even under flow keep increasing showing the adherent character of the cells whereas the negative spots remain flat. The anti EpCAM negative spots again only show a signal increase caused by sedimentation.

Chip Optimization

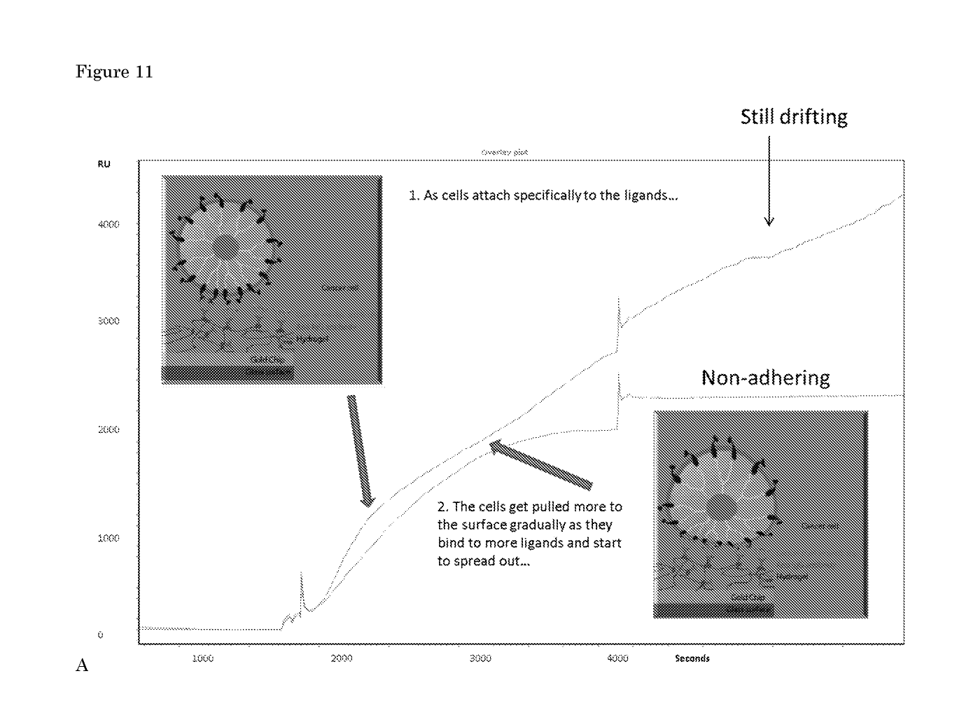

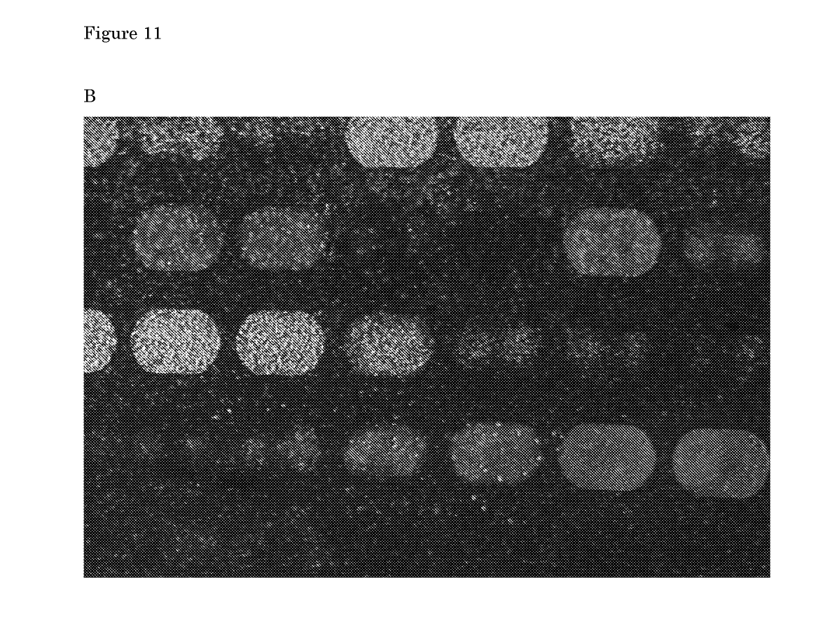

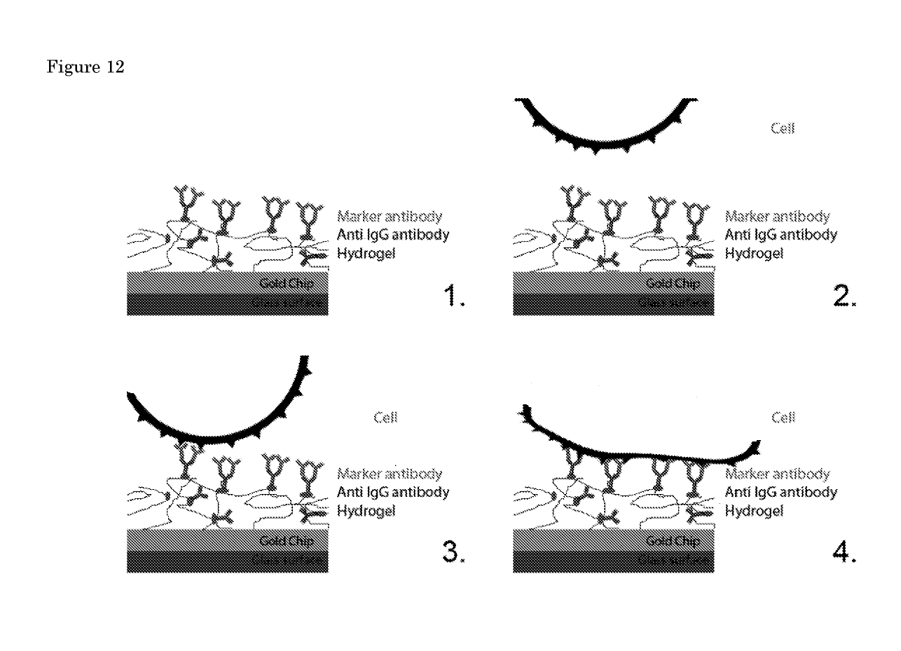

It was noted that BSA had a profound effect on non-specific cell adhering. Spots that were immobilized with 1% BSA in sodium acetate had a considerably lower amount of non-specifically sticking cells as opposed to spots that were covered with a lower concentration of BSA or with just sodium acetate buffer. As such the decision was made to also cover the chips after immobilization with a 1% BSA solution in order to deactivate the remaining bare areas of the chip, this was done in addition to the 2-Aminoethanol step. The chips that used this protocol turned out to have much less non-specific binding as can be seen when comparing FIGS. 10 and 11A, wherein the signals of adherent and non-adherent cells are both depicted. The curves show a lower amount of non-specific sticking on the negative spots in FIG. 10 and even a slight decrease in signal when flow is restarted. FIG. 11B shows the signal intensity for multiple spots, specific interactions show more intense SPR shifts. FIG. 12 shows a schematic representation of adherent cell binding to an SPR chip.

Measurements of HS578T, MCF7 and SKBR3 cells showed similar adherent behavior with EpCAM expression on the cell surface (not shown here).

### Example 6

### Ig-Opsonized Platelets

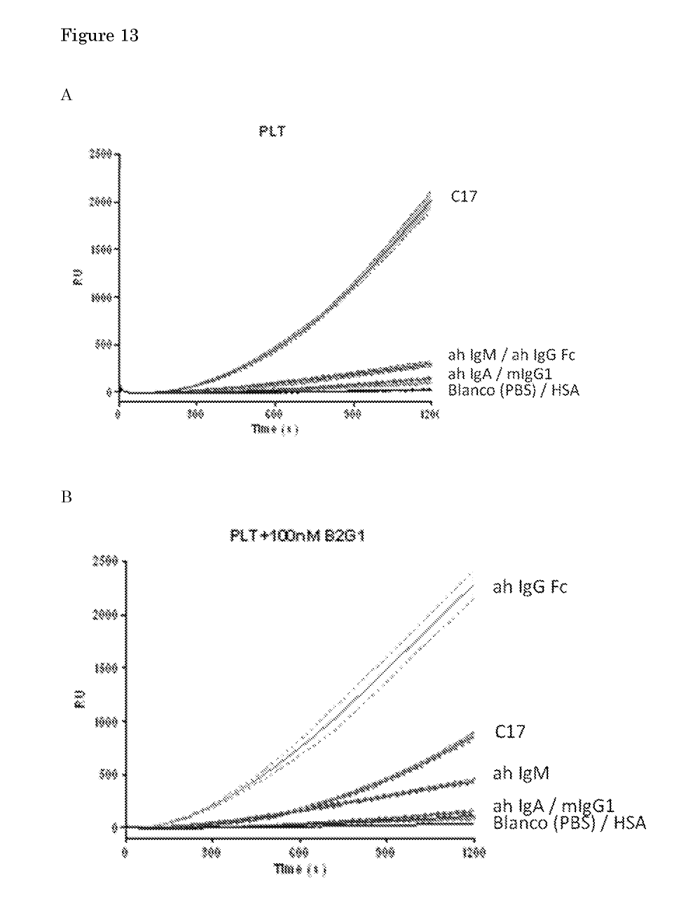

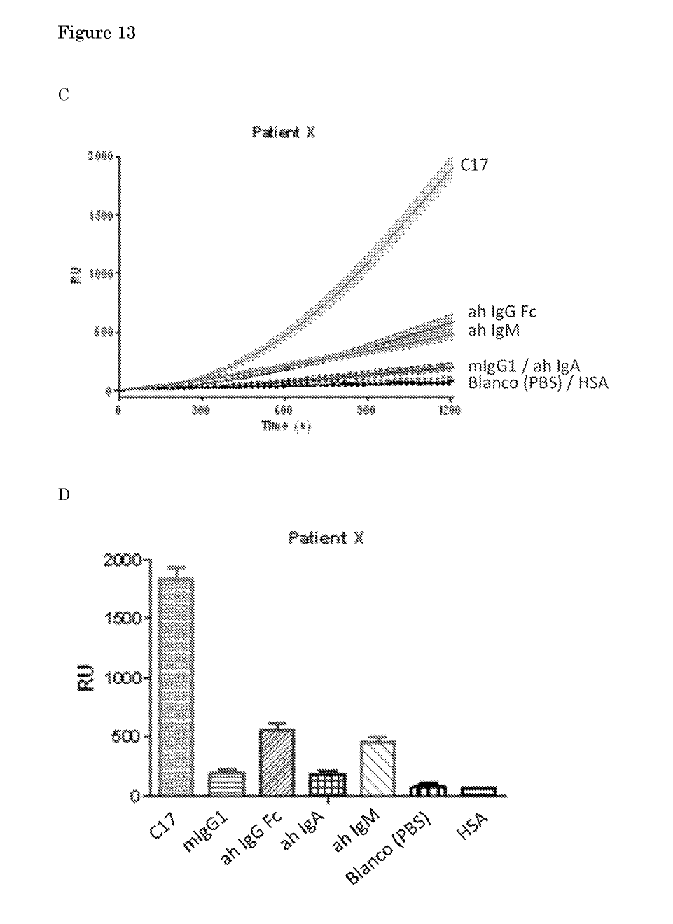

Control platelets and platelets from a patient with thrombocytopenia were used. Platelets from a patient with thrombocytopenia were isolated and injected over the chip to determine if the reduced number of thrombocytes is due to the presence of autoantibodies recognizing the platelets. Isolated patients platelets were directly flowed over a SensEye (Ssense, Enschede, The Netherlands) hosting recombinant anti-HPA1a antibody (B2G1) recognizing the HPA1a epitope on GPIIbIIIa to opsonize platelets. Platelets with and without anti-platelet antibodies are recognized in real-time using IBIS MX96 (IBIS Technologies, Enschede, The Netherlands) (FIG. 13). SPR experiments were performed as described in Example 1. Anti-Immunoglobulin isotypes (ahIgG, ahIgA, and ahIgM), C17 (recognizing platelet gpIIbIIIa), positive control), IgG isotype (mIgG1, negative control) and HSA (control) were coupled to different spots. In addition blank control spots were used. The platelet suspensions was passed into and through the flow chamber with a flow speed of 150 μl/s. After stopping the flow for two minutes, flow was resumed at 20 μl/s in the backward direction.

Control platelets only show a positive signal for positive control C17 spots (FIG. 13A), whereas anti-HPA1a antibody-opsonized platelets in addition bound to ant-IgA and anti-IgM spots (FIG. 13B). Platelets from a patient with thrombocytopenia responses were significantly higher for IgG and IgM, not IgA (FIGS. 13C and 13D).

This example shows that even in thrombocytopenia patients with very low number of platelets (FIG. 13D), when current methods (monoclonal antibody immobilization of platelet antigens (MAIPA), and platelet immunofluorescence test (PIFT)) fail due to the low platelet counts, auto- and/or alloantibodies can be detected and quantified using the described methods.

1. A surface plasmon resonance (SPR) method for detecting a cell surface molecule, the method comprising:
a) providing a sensor comprising a sensor surface, said sensor surface comprising a transparent element covered with a metal film of at most 100 nm thick and at least one binding compound immobilized to said metal film,
b) allowing a liquid cell sample to flow to and along the sensor surface,
c) temporarily reducing shear rate of said liquid sample to allow cells and/or fragments thereof in said liquid sample to bind said binding compound,
d) removing unbound cells or non-specifically bound cells and/or fragments thereof, and
e) determining the ratio of a relative change in refractive index after removing said unbound or non-specifically bound cells and/or fragments thereof and a relative change in refractive index of light after allowing sedimentation and/or binding of said cells and/or fragments thereof to said binding compound, wherein said relative change in refractive index after removing said unbound or non-specifically bound cells and/or fragments thereof is measured after step d) and said relative change in refractive index of light after allowing sedimentation and/or binding of said cells and/or fragments thereof to said binding compound is measured before step d) is performed.

2. Method according to claim 1, wherein said sensor surface is located below said liquid sample.

3. The method according to claim 1, wherein in step c) flow of said liquid sample is essentially stopped.

4. The method according to claim 1, wherein step d) comprises allowing said liquid cell sample to flow along the sensor surface and wherein said liquid sample in step d) has a direction of movement opposite to the direction of movement of said liquid sample in step b).

5. The method according to claim 1, further comprising determining the number of cells and/or fragments thereof in said liquid sample.

6. The method according to claim 1, wherein said at least one binding compound is selected from the group consisting of an antibody or a part thereof, a receptor or cellular ligand, such as a T-cell receptor or Human Leukocyte Antigen (HLA), a DARPin, an aptamer and a lectin.

7. The method according to claim 1, wherein the sensor surface comprises a plurality of binding compounds.

8. The method according to claim 1, wherein said method further comprises disrupting cells bound to said at least one binding compound and adding at least one second binding compound to said disrupted cells or parts thereof bound to said at least one binding compound.

9. The method according to claim 1, wherein said liquid sample comprises cells and/or fragments selected from the group consisting of red blood cells, platelets, microvesicles, white blood cells, non-solid tumor cells, hybridoma cells, stem cells, progenitor cells, bacterial cells, and fungal cells.

10. The method according to claim 1, further comprising detecting at least one antibody in said liquid sample, wherein at least one binding compound is an antibody or a part thereof, or a protein specific for a blood group antigen and at least one binding compound is a blood group antigen, wherein a change in refractive index is measured before binding of cells to said antibody or a part thereof, or protein specific for a blood group antigen, and a change in refractive index is measured both after binding of said cells to said antibody or a part thereof, or protein specific for a blood group antigen but before removing unbound or non-specifically bound cells, and after removing said unbound or non-specifically bound cells.

11. The method according to claim 1, further comprising adding a second binding compound competing with said at least one binding compound for binding to the cell surface molecule.

12. A surface plasmon resonance (SPR) imaging sensor comprising:
a transparent element covered with a metal film of at most 100 nm thick,
a sensor surface comprising a plurality of spots, each spot comprising at least one binding compound immobilized to said sensor surface, wherein said sensor surface comprises a plurality of binding compounds selected from the group consisting of antibodies specific for a blood group antigen and proteins, DARPins, aptamers, and lectins, capable of binding specifically binding a blood group antigen.

13. The SPR sensor according to claim 12, wherein the sensor surface further comprises a plurality of blood group antigens.

14. A surface plasmon resonance (SPR) measuring system comprising the SPR sensor according to claim 12.

15. The SPR measuring system of claim 14, wherein the SPR sensor further comprises a plurality of blood group antigens.

16. A surface plasmon resonance (SPR) method for detecting a cell surface molecule, the method comprising:
flowing a liquid cell along a sensor surface of a sensor, the sensor surface comprising a transparent element covered with a metal film of at most 100 nm thick and at least one binding compound immobilized to the metal film,
temporarily reducing shear rate of the liquid sample to allow cells and/or fragments thereof in the liquid sample to bind the at least one binding compound,
determining a relative change in refractive index of light after allowing sedimentation and/or binding of the unbound cells, non-specifically bound cells and/or fragments thereof to the binding compound
removing unbound cells, non-specifically bound cells, and/or fragments thereof from the liquid sample, and
afterwards, determining the ratio of a relative change in refractive index after removing the unbound cells, non-specifically bound cells, and/or fragments thereof.

17. The SPR method according to claim 16, further comprising:
determining the number of cells and/or fragments thereof in the liquid sample.

18. The SPR method according to claim 16, wherein the at least one binding compound is selected from the group consisting of an antibody, an antibody fragment, a receptor, a cellular ligand, a T-cell receptor, a human leukocyte antigen, a DARPin, an aptamer, and a lectin.

19. The SPR method according to claim 16, wherein the sensor surface comprises a plurality of binding compounds.

20. The SPR method according to claim 16, further comprising:
disrupting cells bound to the at least one binding compound, and
adding at least one second binding compound to the disrupted cells or parts thereof bound to the at least one binding compound.
